(19) United States
(12) Patent Application Publication
(10) Pub. No.: US 2025/0012730 A1
(43) Pub. Date: Jan. 9, 2025

(54) Apparatus And Method For MultiPhase Flowable Medium Analysis

(71) Applicant: AI Exploration Ltd, Edinburgh (GB)

(72) Inventor: Stylianos-Alexios Tsekenis

(73) Assignee: AI Exploration Ltd, Edinburgh, SC (GB)

(21) Appl. No.: 18/570,553

(22) PCT Filed: Jul. 15, 2022
(86) PCT No.: PCT/GB2022/051838

§ 371 Date: Dec. 14, 2023

(30) Foreign Application Priority Data
Jul. 16, 2021 (GR) 20210100480
Jul. 29, 2021 (GB) 2110916.0

(51) Int. Cl. G01N 21/85; G01N 21/35; G01N 33/28

(52) U.S. Cl. CPC G01N 21/8507; G01N 21/35; G01N 33/2823; G01N 2021/8528

(57) ABSTRACT

An apparatus for determining one or more parameters of a multiphase flowable medium flowing in a flow direction through a conduit. The multiphase flowable medium comprises at least a water phase. The apparatus first comprises a probe body configured to extend from a wall of a conduit, such as a wellbore production tubing, into a multiphase flowable medium flowing therethrough. The probe body defines a plurality of sensing locations, each for a different portion of the multiphase flowable medium and spaced in a direction having at least a component transverse to the flow direction. Each sensing location is provided with at least one source configured to emit infrared radiation into the multiphase flowable medium, and at least one photodetector configured to detect infrared radiation received from the at least one source via the multiphase flowable medium.

### STATEMENT OF RELATED APPLICATIONS

The present application is filed under 35 U.S.C. § 371 and claims the benefit of PCT/GB2022/051838. That application is titled “Apparatus and Method for MultiPhase Flowable Medium Analysis,” and was filed Jul. 15, 2022. That application has published as WO 2023/285836.

The PCT application claimed priority to GR 20210100480 filed with the Hellenic Industrial Property Organization on Jul. 16, 2021.

The PCT application also claimed priority to GB 2110916.0 filed Jul. 29, 2021 with the UK Intellectual Property Office. That application has published as GB 2609050.

Each of these applications is incorporated herein in its entirety by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### FIELD OF THE INVENTION

The present invention relates to a method and apparatus for determining one or more parameters of a multiphase flowable medium, for example a flowable medium comprising one or more hydrocarbons and water.

### BACKGROUND TO THE INVENTION

When hydrocarbons flow from a subsea well, through a pipe, often hydrocarbons and water flow together. As a subsea well is used, the proportion of water typically increases. At a certain point, it becomes uneconomical to continue hydrocarbon production using the well. Thus, it is useful to determine the proportion of water flowing from the subsea well, through the pipe.

It is in this context that the present invention has been devised.

### SUMMARY OF THE INVENTION

In accordance with an aspect of the present invention, there is provided an apparatus for use in determining one or more parameters of a multiphase flowable medium flowing in a flow direction through a conduit. The multiphase flowable medium typically comprises at least a water phase. The apparatus comprises: a probe body for extending from a wall of a conduit, into a multiphase flowable medium flowing therethrough. The probe body defines a plurality of sensing locations, each for a different portion of the multiphase flowable medium. Each sensing location is mutually spaced from any other one of the plurality of sensing locations in a direction having at least a component transverse to the flow direction. Each sensing location is provided with: at least one source configured to emit infrared radiation into the multiphase flowable medium; and at least one photodetector configured to detect infrared radiation received from the at least one source via the multiphase flowable medium.

Thus, the one or more parameters of the multiphase flowable medium can be determined by using infrared measurements from sensing locations at different transverse positions in the conduit. Accordingly, the flow is measured in at least two different transversely spaced positions, and the variation therein is used in determining the one or more parameters. As a result, the one or more parameters can be determined particularly effectively.

By each sensing location being provided with at least one source and at least one photodetector, it will be understood that each sensing location is provided with the infrared radiation emitted from the at least one source, and that the at least one photodetector can be used to detect the infrared radiation received via the multiphase flowable medium at the sensing location, regardless of whether the at least one source and/or the at least one photodetectors themselves are located at the sensing location, or are merely in optical communication therewith via an optical arrangement. In other words, the apparatus comprises, for each of the plurality of sensing locations, the at least one source, and the at least one photodetector. The at least one source is configured to emit infrared radiation into the multiphase flowable medium. The at least one photodetector is configured to detect infrared radiation received from the at least one source via the multiphase flowable medium at the respective sensing location.

The multiphase flowable medium may further comprise a hydrocarbon phase. The probe body may be for insertion through the wall of the conduit.

It will be understood that in some embodiments, there is no need for a plurality of sensing locations, and the probe body may define only a single sensing location provided with at least one source and at least one photodetector. The features described hereinafter can be incorporated into any of the apparatus described herein, unless inherently incompatible.

The one or more parameters may comprise at least one of a water cut, a water holdup and a water in liquid ratio. The one or more parameters may be indicative of a concentration of water in the multiphase flowable medium. The one or more parameters may comprise an oil-cut. It will be understood that the oil-cut may be considered to be indicative of the concentration of water in the multiphase flowable medium since an increase in the concentration of water generally equates to a decrease in the concentration of oil.

The conduit may form part of a hydrocarbon well. In this way, it will be understood that the apparatus may be for determining the one or more parameters of an output multiphase flowable medium, from a hydrocarbon well. The hydrocarbon well may be an oil well, a condensate well, a wet gas well, a dry gas well, or a fracking well.

Each sensing location may be separated from any other one of the plurality of sensing locations by at least one centimetre in the direction having at least a component transverse to the flow direction. Thus, the sensing locations are sufficiently separated that variations in the concentration of the phases between adjacent ones of at least some of the positions of the sensing locations may be observed. The separation may be at least three centimetres. The direction having at least a component transverse to the flow direction may be substantially transverse to the flow direction.

Each sensing location may be separated from any other one of the plurality of sensing locations by less than 30 centimetres in the direction having the component transverse to the flow direction. Thus, the sensing locations are sufficiently closely spaced that at least some adjacent sensing locations may both be within an emulsion region of the multiphase flow. It may be that the plurality of sensing locations are distributed along the probe body such that at least two of the sensing locations are within an emulsion region of the multiphase flow.

It may be that at least one sensing location is within less than 50 centimetres of each other sensing location. It may be that at least one sensing location is within less than 30 centimetres of each other sensing location. It may be that at least one sensing location is within less than a distance equal to 1.5 times the radius of the conduit, from each other sensing location. It may be that at least one sensing location is within less than a distance equal to the radius of the conduit, from each other sensing location.

In some examples, there may be exactly two sensing locations.

The probe body may be configured to extend through a central region of the conduit. At least one of the sensing locations may be for the central region of the multiphase flowable medium flowing through the conduit. It may be that each of the sensing locations is for the central region of the multiphase flowable medium flowing through the conduit. It will be understood that the central region of the multiphase flowable medium is a portion of the multiphase flowable medium away from the wall of the conduit, such as the inner 50% of the cross-sectional area of the conduit, or even the inner 25% of the cross-sectional area of the conduit. For a cylindrical conduit, it may be that the central portion is the inner 50% of the radius of the conduit. Thus, the apparatus can be used to conduct sensing in a central portion of the multiphase flowable medium flowing through the conduit. It may be that at least one sensing location is within a central region of the conduit. At least one sensing location may be near to (e.g. within 10% of a radius of the conduit from) the wall of the conduit. In some examples, a first sensing location is within a central region of the conduit and a second sensing location is near to the wall of the conduit.

It may be that the probe body is arranged to occupy less than 20% of the cross-sectional area of the conduit, such as less than 10% of the cross-sectional area of the conduit. Thus, the use of the apparatus does not provide an unacceptable flow constriction in the conduit. The probe body may be configured to extend from the wall of the conduit, to the central region of the conduit. It may be that the probe body is configured to extend only from a single side of the wall of the conduit.

It may be that at least one of the sensing locations is arranged to be away from the wall of the conduit. It may be that at least one of the sensing locations is arranged to be more than 5 centimetres from the wall of the conduit, such as more than 10 centimetres from the wall of the conduit, for example more than 20 centimetres from the wall of the conduit. It may be that each of the sensing locations is arranged to be away from the wall of the conduit. It may be that each of the sensing locations are arranged to be more than 5 centimetres from the wall of the conduit, such as more than 10 centimetres from the wall of the conduit, for example more than 20 centimetres from the wall of the conduit. Thus, at least one, if not all, of the sensing locations are away from the wall of the conduit, thereby allowing the fluid properties of the multiphase flowable medium flowing therethrough to be determined at or near the central region of the conduit.

At least one of the sensing locations is arranged to be at the wall of the conduit. Thus, the fluid properties of the multiphase flowable medium flowing near the wall of the conduit can be determined. In some examples, it may be that at least one sensing location is arranged to be at the wall of the conduit and at least one further sensing location is arranged to be away from the wall of the conduit.

The apparatus may be arranged to obstruct less than 50% of the cross-sectional area of the conduit, for example less than 20% of the cross-sectional area, such as less than 10% of the cross-sectional area.

The at least one source of each sensing location may be configured to emit infrared radiation in a first water wavelength band, and in a second water wavelength band. Each of the first water wavelength band and the second water wavelength band are typically a region of the infrared spectrum in which the water phase exhibits absorption.

In examples, the at least one source of each sensing location is configured to emit a reduced amount of (e.g. no) infrared radiation in an intermediate wavelength band between the first water wavelength band and the second water wavelength band.

It may be that the at least one source comprises a first source configured to emit infrared radiation in the first water wavelength band and a second source configured to emit infrared radiation in the second water wavelength band. The first source and the second source may each be a narrowband source. A −3 dB bandwidth of one or both of the first source and the second source may be less than 10 nanometres, such as less than 5 nanometres. In some examples, the first source and the second source may each by configured to emit infrared radiation having only a single wavelength. The first source and the second source may each be a laser.

Additionally or alternatively, the at least one photodetector of each sensing location may be configured to detect infrared radiation in the first water wavelength band, and in the second water wavelength band.

Additionally or alternatively, the at least one photodetector of each sensing location may be configured to detect a reduced amount of (e.g. no) infrared radiation in the intermediate wavelength band.

The at least one source may be a broadband thermal source. It may be that a single broadband thermal source is provided for each sensing location. Thus, infrared radiation having all wavelengths of interest can be emitted into the multiphase flowable medium at the sensing location using the single broadband thermal source. A beam-splitter and filter arrangement may be provided to split the detected infrared radiation received from the source via the multiphase flowable medium to a plurality of spectrally-filtered photodetectors. Thus, the source need not be carefully tuned, providing that infrared radiation is emitted which includes at least the required wavelengths. In some examples, it may even be that a single broadband thermal source is provided for multiple sensing locations. A beam splitter may be provided to direct the infrared radiation to multiple sensing locations.

Thus, one or both of the at least one source and/or the at least one photodetector can be matched to the wavelengths of interest for water.

The first water wavelength band and the second water wavelength band may each include one or more wavelengths between 1350 nanometres and 1950 nanometres. Thus, the first and second water wavelength bands together include wavelengths in a region containing two significant absorption peaks in the absorption spectrum for water. It will be understood that there are absorption peaks for water at around 1450 nanometres and 1950 nanometres. It may be that the first water wavelength band and the second water wavelength band each include one or more wavelengths within the same absorption peak. It may be that the first water wavelength band and the second water wavelength band each include one or more wavelengths on the same side of the same absorption peak (i.e. both lower or both higher than a maximum absorption response in the absorption peak).

The first water wavelength band and the second water wavelength band may each include one or more wavelengths between 1850 nanometres and 1950 nanometres. Thus, the first and second water wavelength bands both include wavelengths in a region of the electromagnetic spectrum containing a particularly significant absorption peak for water. The absorption peak at 1950 is particularly suitable due to the lack of interference from hydroxyl groups (ROH) and C—H 1^{st} overtone combination absorption (both exhibited around the 1450 peak). More specifically, both the first and second water wavelength bands may include wavelengths on a lower side of the same absorption peak (i.e. wavelengths in the absorption band, having a value less than the wavelength at the absorption peak). It has been found by the present inventors that the lower side of the absorption peak is typically less affected (shifts in position and intensity of the absorption response) by temperature fluctuations compared to the right side of the absorption peak.

In another example, the first water wavelength band and the second water wavelength band may each include one or more wavelengths between 1450 nanometres and 1650 nanometres. Thus, the upper side of the absorption peak (i.e. wavelengths in the absorption band, having a value greater than the wavelength at the absorption peak) around 1450 nanometres can be used.

The first water wavelength band and the second water wavelength band may each have a −3 dB bandwidth of less than 10 nanometres, for example less than 5 nanometres. Thus, each of the first and second water wavelength bands are considered narrow bands. In some examples, the first water wavelength band and the second water wavelength band may each be associated with substantially a single wavelength.

The first water wavelength band may be separated from the second water wavelength band by less than 50 nanometres. Thus, the first and second water wavelength bands are typically reasonably closely spaced. As a result, extinction effects with a slight wavelength dependency can be ignored as they will be substantially equal at both water wavelength bands. The extinction effects may include absorption from other phases (such as a hydrocarbon phase), as well as Rayleigh scattering effects. Typically, both of the first and second water wavelength bands are associated with the same peak in the absorption spectrum for water. By sensing the absorption of the infrared radiation at different wavelengths associated with different points on the same side of the absorption peak, a relatively large change in the absorption coefficient can be observed, even with only a small change in wavelength.

The apparatus may be configured to adjust a centre wavelength of the first water wavelength band within a first water wavelength range. The apparatus may be configured to adjust a centre wavelength of the second water wavelength band within a second water wavelength range. Thus, even where a narrowband source is used for each of the first and second water wavelength bands, the wavelength band can be tuned to match any slight variations in the absorption spectrum due to variations in the local environment of the multiphase flowable medium. Furthermore, the signal to noise ratio of the signal can be optimised in the case of very low or very high concentrations of water in the multiphase flowable medium by adjusting the centre wavelength of one or both of the first and second water wavelength band.

For example, for a very low concentration of water, it is important that whichever of the first and second water wavelength bands expected to produce a response closest to the response at the absorption peak, is adjusted to have a wavelength closer to the wavelength of the absorption peak, than would be the case for a normal or even very high concentration of water in the multiphase flowable medium. As a result, the sensitivity of the apparatus is sufficient to detect the presence of water, even at very low concentrations because the first and second water wavelength bands can be located sufficiently close to the absorption peak to allow absorption to be detected, even where the concentration of water is low. It may be that the first and second water wavelength bands are brought particularly close together, so that the ratio between them would not be expected to be so high. Therefore, the measurement of the concentration of water at very low concentrations would be less precise than were the first and second water wavelength bands to be more widely spaced.

For a very high concentration of water, it is important that the concentration can be determined with sufficient precision. Owing to the larger concentration of water, it would be expected that the water could be easily detected over the system noise and other interferences, even at regions in the absorption band, spaced from the peak. Accordingly, the centre wavelength of the first and second water wavelength bands can be adjusted such that a region of the absorption peak is used resulting in a large difference between the response for the first and second water wavelength bands, whilst the first and second water wavelength bands are still sufficiently closely located that other wavelength-dependent variations in the absorption spectrum can be approximated as substantially wavelength independent. As a result of the response at the first water wavelength band being so different to the response at the second water wavelength band, the ratio between the two responses can be more extreme than in other regions of the absorption band, allowing the concentration of water to be determined with greater precision.

The first water wavelength range and the second water wavelength range may each be less than 50 nanometres, for example less than 30 nanometres.

Where the multiphase flowable medium comprises a hydrocarbon phase, the at least one source of each sensing location may be configured to emit infrared radiation in a first hydrocarbon wavelength band, and in a second hydrocarbon wavelength band. The first hydrocarbon wavelength band is typically in a region of the infrared spectrum in which the hydrocarbon phase exhibits absorption. The second hydrocarbon wavelength band may also be in a region of the infrared spectrum in which the hydrocarbon phase exhibits absorption. Thus, measurements of the electromagnetic radiation received in the first and second hydrocarbon wavelength bands can be used to determine one or more parameters related to the hydrocarbon phase of the multiphase flowable medium, such as a parameter indicative of a concentration of the hydrocarbon substance in the multiphase flowable medium.

The at least one source of each sensing location may be configured to emit a reduced amount of (e.g. no) infrared radiation in an intermediate wavelength band between the first hydrocarbon wavelength band and the second hydrocarbon wavelength band.

It may be that the at least one source comprises a first hydrocarbon source configured to emit infrared radiation in the first hydrocarbon wavelength band and a second hydrocarbon source configured to emit infrared radiation in the second hydrocarbon wavelength band. The first hydrocarbon source and the second hydrocarbon source may each be a narrowband source. A −3 dB bandwidth of one or both of the first hydrocarbon source and the second hydrocarbon source may be less than 10 nanometres, such as less than 5 nanometres. In some examples, the first hydrocarbon source and the second hydrocarbon source may each by configured to emit infrared radiation having only a single wavelength. The first hydrocarbon source and the second hydrocarbon source may each be a laser.

In some examples, the at least one source comprises a single hydrocarbon source configured to emit infrared radiation in the first hydrocarbon wavelength band and in the second hydrocarbon wavelength band and an optical filter to attenuate infrared radiation outside the first hydrocarbon wavelength band and the second hydrocarbon wavelength band. The single hydrocarbon source may be a superluminescent light emitting diode (SLED).

Additionally or alternatively, the at least one photodetector of each sensing location may be configured to detect infrared radiation in the first hydrocarbon wavelength band, and in the second hydrocarbon wavelength band.

Additionally or alternatively, the at least one photodetector of each sensing location may be configured to detect a reduced amount of (e.g. no) infrared radiation in the intermediate wavelength band.

The first hydrocarbon wavelength band and the second hydrocarbon wavelength band may each include one or more wavelengths between 1550 nanometres and 1850 nanometres. Thus, the first and second hydrocarbon wavelength bands may include wavelengths in or around the absorption peak for hydrocarbons around 1750 nanometres. It may be that the first hydrocarbon wavelength band and the second hydrocarbon wavelength band each include one or more wavelengths on the same side of the same absorption peak (i.e. both lower or both higher than a maximum absorption response in the absorption peak).

The first hydrocarbon wavelength band and the second hydrocarbon wavelength band may each include one or more wavelengths between 1620 nanometres and 1750 nanometres. Thus, both the first and second hydrocarbon wavelength bands include wavelengths on or below the lower side of the absorption peak (i.e. wavelengths in the absorption band, having a value less than the wavelength at the absorption peak) for hydrocarbons around 1750 nanometres.

The first hydrocarbon wavelength band may include one or more wavelengths between 1690 and 1780 nanometres. The first hydrocarbon wavelength band may include one or more wavelengths between 1690 and 1750 nanometres. Thus, the first hydrocarbon wavelength band may include wavelengths forming part of the absorption peak for hydrocarbons around 1750 nanometres. The second hydrocarbon wavelength band may include one or more wavelengths between 1620 and 1660. Thus, the second hydrocarbon wavelength band may include wavelengths not forming part of the absorption peak for hydrocarbons around 1750 nanometres, for example having a wavelength below the absorption peak for hydrocarbons around 1750 nanometres, such as having a wavelength associated with less absorption compared with the absorption expected for hydrocarbons within the absorption peak around 1750 nanometres.

The first hydrocarbon wavelength band and the second hydrocarbon wavelength band may each have a −3 dB bandwidth of less than 10 nanometres, for example less than 5 nanometres. Thus, each of the first and second hydrocarbon wavelength bands are considered narrow bands. In some examples, the first hydrocarbon wavelength band and the second hydrocarbon wavelength band may each be associated with substantially a single wavelength. In some examples, it may be that only one of the first and second hydrocarbon wavelength bands has the spectral range limitations described hereinbefore.

The first hydrocarbon wavelength band may be separated from the second hydrocarbon wavelength band by more than 20 nanometres. The first hydrocarbon wavelength band may be separated from the second hydrocarbon wavelength band by more than 30 nanometres. The first hydrocarbon wavelength band may be separated from the second hydrocarbon wavelength band by less than 500 nanometres, such as less than 200 nanometres.

The apparatus may be configured to adjust a centre wavelength of the first hydrocarbon wavelength band within a first hydrocarbon wavelength range. The apparatus may be configured to adjust a centre wavelength of the second hydrocarbon wavelength band within a second hydrocarbon wavelength range. Thus, even where a narrowband source is used for each of the first and second hydrocarbon wavelength bands, the wavelength band can be tuned to match any slight variations in the absorption spectrum due to variations in the local environment of the multiphase flowable medium. Furthermore, as discussed previously with reference to adjustment of the centre wavelength of the water wavelength bands, either of sensitivity or precision of the concentration determination can be optimised by adjustment of the centre wavelengths of one or both of the first and second hydrocarbon wavelength bands.

The first hydrocarbon wavelength range and the second hydrocarbon wavelength range may each be less than 50 nanometres, for example less than 30 nanometres.

The at least one source may comprise: a first source configured to emit infrared radiation in substantially only the first water wavelength band; a second source configured to emit infrared radiation in substantially only the second water wavelength band; a third source configured to emit infrared radiation in substantially only the first hydrocarbon wavelength band; and a fourth source configured to emit infrared radiation in substantially only the second hydrocarbon wavelength band. The at least one photodetector may comprise: a first photodetector configured to detect infrared radiation in the first water wavelength band and the second water wavelength band; and a second photodetector configured to detect infrared radiation in the first hydrocarbon wavelength band and the second hydrocarbon wavelength band.

Each sensing location may be further provided with a source optical arrangement configured to combine the infrared radiation emitted from a plurality of sources (e.g. the four sources) to be directed together through the multiphase flowable medium at the sensing location. Each sensing location may be further provided with a detector optical arrangement configured to split the infrared radiation received from the multiphase flowable medium into a first wavelength band to be directed to the first photodetector and a second wavelength band to be directed to the second photodetector. Thus, the infrared radiation passes through exactly the same region of the multiphase flowable medium, even though it originated from different sources, and may be directed to one of two different photodetectors depending on the wavelength of the radiation.

The source optical arrangement may be configured to provide a collimated beam of infrared radiation for direction through the multiphase flowable medium at the sensing location. The detector optical arrangement may be configured to split the collimated beam of infrared radiation.

The source optical arrangement may comprise three beam combiners. The detector optical arrangement may comprise a beamsplitter.

A source reflector may be provided in an optical path between the at least one source and the multiphase flowable medium at the sensing location. A detector reflector may be provided in an optical path between the multiphase flowable medium at the sensing location and the at least one photodetector. At least one of the source reflector and the detector reflector may be a mirror arranged to reflect the incident radiation from the at least one source, received via the multiphase flowable medium for the detector reflector, by, for example, 60 degrees to 120 degrees, such as substantially 90 degrees (e.g. 90 degrees). Thus, a more compact arrangement for the apparatus can be provided, which need not take up so much room in the direction in which the infrared radiation passes through the multiphase flowable medium.

The at least one source may be a plurality of sources. An emission signal from each source may be modulated on a respective modulation frequency. One or more detection signals output from the photodetector may be demodulated using the respective modulation frequencies. Thus, blackbody background radiation, measurement noise and DC offsets are suppressed.

The demodulation may be in the form of an in-phase demodulation and a quadrature demodulation, combined together to provide the output signal. Thus, the effect of any phase shift introduced during transmission of the infrared radiation through the multiphase flowable medium can be accounted for.

The probe body typically defines at least one channel through which the multiphase flowable medium is arranged to flow. The plurality of sensing locations are typically provided at the at least one channel. The at least one channel may be a plurality of channels, one each for each of the plurality of sensing locations. Each channel may be arranged to extend in a flow-wise direction.

A minimum width of the or each channel, through which infrared radiation from the at least one source must pass to be detected by the at least one photodetector may be less than five millimetres. Thus, the channel is sufficiently small that not all of the infrared radiation will be absorbed and/or scattered or otherwise lost by passage through the channel containing the multiphase flowable medium.

A first channel of the plurality of channels may be defined at a first side of the probe body. A second channel of the plurality of channels may be defined at a second side of the probe body, the second side opposite the first side. Thus, a more compact arrangement of the probe body can be provided, because the optical components associated with each channel can be interleaved.

In some examples, at least one (e.g. each) channel is a laterally bounded channel, that is to say that the at least one channel is laterally bounded by the probe body on all sides. In other words, the at least one channel extends through an internal region of the probe body. In some examples, at least one (e.g. each) channel is open on at least one lateral side, that is to say that the at least one channel is laterally unbounded on at least one side. It will be understood that the channels are typically open at a channel inlet and a channel outlet to allow the multiphase flowable medium to flow through the channel.

At least one (e.g. each) channel may be provided with a convergent region forming a mouth of the channel. Thus, a larger portion of the flow is directed through the channel than would be the case were the channel not to be provided with a convergent mouth. It will be understood that the presence of the convergent region creates a high pressure region in the fluid flow, such that a speed of flow through the channel is greater than a speed of flow in a region of the conduit immediately upstream of the channel. By conveying a larger portion of the flow through the channel, a more representative sample of that portion of the flow can be analysed using the apparatus. Furthermore, due to the increased flow speed through the channel, a likelihood of blockage of the channel can also be reduced.

The probe body may further comprise a mounting portion for mounting the probe body relative to the wall of the conduit. Thus, the probe body can be easily secured in place within the conduit using the mounting portion.

It may be that the probe body is provided with one or more flow modification features, such as having a teardrop cross-section, or a helical strake.

The probe body may be configured to extend perpendicularly from the wall of the conduit. In some examples, where the conduit runs substantially horizontally, the probe body may be configured to extend vertically within the conduit, for example, vertically downward from an upper wall of the conduit.

The apparatus may further comprise a controller. The controller may comprise one or more processors and a non-transient computer-readable memory storing instructions. The instructions, when executed by the one or more processors may cause the controller to operate one or more components of the apparatus as described herein. It will be understood that in some examples the one or more processors may be located in a single unit. In other examples, where the one or more processors is a plurality of processors, the controller may be distributed, which is to say that at least one of the plurality of processors may be located separated from at least one other of the plurality of processors.

The controller may be configured to determine an output indicative of a concentration of water in the multiphase flowable medium at each sensing location. The output indicative of the concentration of water may be determined based on an output signal from the at least one photodetector for the respective channel. It may be that the output indicative of the concentration of water for the respective channel is determined in dependence on an output signal from a plurality of photodetectors for the respective channel.

The controller may be configured to determine the output indicative of the concentration of water in dependence on determining a relative water response parameter. The controller may be configured to determine the relative water response parameter in dependence on a first output signal from the at least one photodetector, indicative of infrared radiation received in the first water wavelength band, and a second output signal from the at least one photodetector, indicative of infrared radiation in the second water wavelength band. The relative water response parameter may be a water ratio. For example, the water ratio may be determined as the ratio between the first output signal and the second output signal.

The controller may be configured to determine the output indicative of the concentration of water in dependence on determining a relative hydrocarbon response parameter. The controller may be configured to determine the relative hydrocarbon response parameter in dependence on a third output signal from the at least one photodetector, indicative of infrared radiation received in the first hydrocarbon wavelength band, and a fourth output signal from the at least one photodetector, indicative of infrared radiation received in the second hydrocarbon wavelength band. The controller may be further configured to determine the output indicative of the concentration of water in further dependence on determining a combined relative parameter. The controller may be configured to determine the combined relative parameter in dependence on the relative water response parameter and the relative hydrocarbon response parameter. The relative hydrocarbon response parameter may be a hydrocarbon ratio. The hydrocarbon ration may be determined as the ratio between the third output signal and the fourth output signal. The combined relative parameter may be a combined ratio. The combined ratio may be determined as the ratio between the relative water response parameter and the relative hydrocarbon response parameter, such as the ratio between the water ratio and the hydrocarbon ratio.

In accordance with a further aspect of the present invention, there is provided apparatus for use in determining an output indicative of a concentration of a substance in a multiphase flowable medium flowing in a flow direction through a conduit. The multiphase flowable medium comprises a plurality of phases. The apparatus comprises: a probe body extending from a wall of a conduit, into a multiphase flowable medium flowing therethrough (through the conduit). The probe body defines a sensing location provided with: at least one source configured to emit infrared radiation into the multiphase flowable medium in a first wavelength band and a second wavelength band. Each of the first wavelength band and the second wavelength band are in a region of the infrared spectrum in which the substance exhibits absorption. The sensing location is further provided with at least one photodetector configured to detect infrared radiation received from the at least one source via the multiphase flowable medium in the first wavelength band and the second wavelength band, and a controller configured to determine an output indicative of a concentration of a substance in the multiphase flowable medium, based on determining a relative absorption parameter. The controller is configured to determine the relative absorption parameter in dependence on a first output signal from the at least one photodetector, indicative of infrared radiation received in the first wavelength band, and a second output signal from the at least one photodetector, indicative of infrared radiation in the second wavelength band. The first wavelength band and the second wavelength band are each at different respective regions of the infrared spectrum on the same absorption peak of the substance.

Thus, by using the same peak, the variation in the extinction of the radiation from phenomena other than absorption by the substance can be reduced even where those phenomena exhibit slightly wavelength dependent variation. By using different parts of the peak, there is still a sufficient difference in the absorption to be able to detect and determine an indication of the concentration of the substance in the multiphase flowable medium.

It will be understood that an absorption peak may be defined as the region of the electromagnetic spectrum in which the absorption coefficient for the substance includes at least one local maximum between two local minima. It may be that each of the first and second wavelength bands may be between the local maximum and a one of the two local minima defining the extent of the absorption peak. The first and second wavelength bands may be between the local maximum and a first of the two local minima, the first local minimum having a wavelength lower than the local maximum.

The wavelength of at least one (e.g. both) of the first and second wavelength bands may be variable, in dependence on an indication of the concentration of the substance (e.g. an expected concentration of the substance). Specifically, it may be that at least one of the first and second wavelength bands are varied to give a higher response (e.g. to be at a higher wavelength, closer to the local maximum) where an expected concentration of the substance is low, and/or are varied to give a lower response (e.g. to be at a lower wavelength, closer to the local minima) where an expected concentration of the substance is high.

As previously noted, the relative absorption parameter may be a ratio. The ratio may be determined as the ratio between the first output signal and the second output signal.

The substance may be water.

The present invention extends to a conduit for having multiphase flowable medium flowing therethrough, and having a probe body of the apparatus as described hereinbefore, extending from, and secured to, a wall of the conduit.

Viewed from another aspect, the present invention provides a method of determining one or more parameters of a multiphase flowable medium flowing in a flow direction through a conduit. The multiphase flowable medium comprises at least a water phase. The method comprises: providing the apparatus as described hereinbefore, of any of the types defining a plurality of sensing locations; controlling the at least one source of each sensing location to emit infrared radiation into the multiphase flowable medium at the respective sensing location; receiving an output signal from the at least one photodetector of each sensing location; and determining one or more parameters of the multiphase flowable medium in dependence on the received output signals.

Indeed, the present invention extends to a method of determining one or more parameters of a multiphase flowable medium flowing in a flow direction through a conduit. The multiphase flowable medium comprises at least a water phase. The method comprises: providing any of the forms of apparatus as described hereinbefore; controlling the at least one source to emit infrared radiation into the multiphase flowable medium at a sensing location; receiving an output signal from the at least one photodetector of the sensing location; and determining one or more parameters of the multiphase flowable medium in dependence on the received output signal.

Viewed from yet another aspect, the present invention extends to a method of determining an output indicative of a concentration of a substance in a multiphase flowable medium flowing in a flow direction through a conduit. The multiphase flowable medium comprises a plurality of phases. The method comprises: providing the apparatus as described hereinbefore; controlling the at least one source to emit infrared radiation into the multiphase flowable medium, at a sensing location, in the first wavelength band and the second wavelength band; receiving, from the at least one photodetector, a first output signal indicative of infrared radiation received from the at least one source, via the multiphase flowable medium at the sensing location, in the first wavelength band, and a second output signal indicative of infrared radiation received from the at least one source, via the multiphase flowable medium at the sensing location, in the second wavelength band; and determining an output indicative of a concentration of a substance in the multiphase flowable medium, based on determining a relative absorption parameter. The relative absorption parameter is determined in dependence on the first output signal and the second output signal. The relative absorption parameter may be an absorption ratio. The absorption ratio may be determined as a ratio between the first output signal and the second output signal.

Viewed from a yet further aspect, the present invention extends to a method of determining one or more parameters of a multiphase flowable medium flowing in a flow direction through a conduit. The multiphase flowable medium comprises at least a water phase. The method comprises: receiving a plurality of concentration inputs, each indicative of a concentration of at least one substance of the multiphase flowable medium in the conduit at a respective position within the conduit, each position mutually spaced in a direction transverse to the flow direction; and determining one or more parameters of the multiphase flowable medium in dependence on the plurality of concentration inputs.

Thus, multiple values of local concentration within the conduit can be used to provide an indication of the concentration variation within a cross-section of the conduit, and therefore used to provide an indication of one or more likely parameters relating to the whole multiphase flowable medium in that cross-section through the conduit.

The method may comprise outputting the determined one or more parameters.

The one or more parameters may include a phase amount indicator, indicative of an amount of at least one phase of the multiphase flowable medium flowing through the conduit.

The phase amount indicator may be indicative of an amount of water flowing through the conduit.

The method may further comprise determining a gradient indicator indicative of an emulsion concentration gradient in dependence on the plurality of concentration inputs. Thus, by determining an indication of concentration at two different locations in a cross-section through the conduit, a linear gradient for the progression of the concentration in the emulsion can be determined.

The method may further comprise determining an emulsion-oil phase change position and an emulsion-water phase change position in the conduit in dependence on the gradient indicator.

The phase amount indicator may be determined in dependence on the emulsion-oil phase change position and the emulsion-water phase change position.

The plurality of concentration inputs may be determined using the methods described hereinbefore. Indeed, any of the methods described herein may be implemented with any of the apparatus described herein. Similarly, the present disclosure extends to methods of using any of the apparatus as described.

### DESCRIPTION OF THE DRAWINGS

An example embodiment of the present invention will now be illustrated with reference to the following Figures in which:

FIGS. 1a and 1b illustrate an apparatus for use in determining one or more parameters of a multiphase flowable medium in accordance with an aspect of the present invention, viewed from different directions;

FIG. 2 shows the apparatus of FIGS. 1a and 1b, in use within a conduit;

FIG. 3 shows an example of a schematic illustration of an optical and electrical circuit of an apparatus for use in determining one or more parameters of a multiphase flowable medium in accordance with an aspect of the present invention;

FIG. 4 shows an absorption spectrum of water;

FIG. 5 shows an absorption spectrum of two different hydrocarbons;

FIG. 6 shows measurement bands relative to the absorption spectrum of water in accordance with an aspect of the present invention;

FIG. 7 shows measurement bands relative to the absorption spectrum of crude oil in accordance with an aspect of the present invention;

FIG. 8 is a graphical illustration of an optical arrangement for use in the circuit of FIG. 3;

FIG. 9 shows a schematic illustration of a further circuit for use in the circuit of FIG. 3;

FIG. 10 shows an example phase arrangement within a multiphase flowable medium;

FIG. 11 is a graph showing a concentration of water (y-axis) with the distance through the pipe in the d direction, for the phase arrangement of FIG. 10;

FIGS. 12 to 14 are flow diagrams illustrating methods of determining parameters of the multiphase flowable medium in accordance with aspects of the present inventions; and

FIG. 15 is a schematic illustration of apparatus comprising a controller in accordance with an aspect of the present invention.

### DETAILED DESCRIPTION OF AN EXAMPLE EMBODIMENT

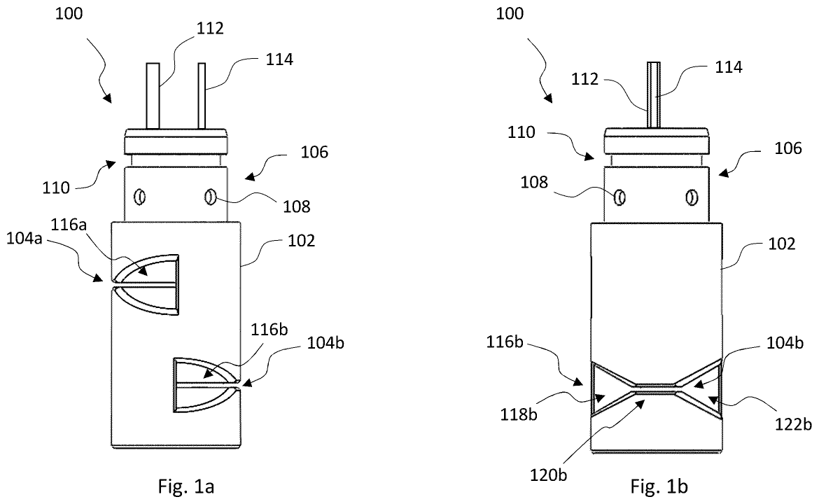

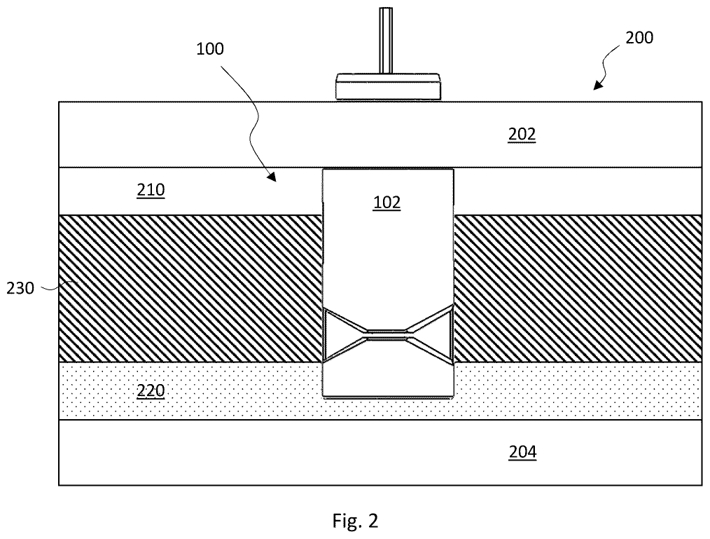

FIGS. 1a and 1b illustrate an apparatus for use in determining one or more parameters of a multiphase flowable medium in accordance with an aspect of the present invention, viewed from different directions. FIG. 1a shows an apparatus 100 for use in determining one or more parameters of a multiphase flowable medium (not shown in FIGS. 1a, 1b). The apparatus 100 comprises a probe body 102. The probe body 102 has a substantially cylindrical cross-section and provides a housing of the apparatus 100, for housing sensitive electrical and/or optical components of the apparatus 100. The probe body 102 further provides at least one sensing location 104a, 104b, in the form of a first sensing location 104a and a second sensing location 104b. The first sensing location 104a is spaced from the second sensing location 104b. Each of the sensing locations 104a, 104b is provided at a respective channel 116a, 116b defined in the housing body 102 at the sensing locations 104a, 104b. The probe body 102 extends from a mounting portion 106. The mounting portion 106 includes a plurality of mechanical connectors 108 for mechanically securing the apparatus 100 to a wall of a conduit (not shown). The mounting portion 106 further includes a seal portion 110 which, in combination with a sealing member (not shown) is used to provide a fluid-tight seal when the apparatus 100 is installed within the conduit.

As will be described further hereinafter, the sensing locations 104a, 104b are provided with one or more sources for emitting infrared radiation into the channel defined in the probe body 102 and one or more photodetectors for detecting infrared radiation received from the respective sources via the channel defined in the probe body 102. Power and communications are provided to the apparatus 100 via wired connections 112, 114. Specifically, power can be provided to the apparatus 100 via a wired power connection 112. Control signals to the components of the apparatus 100 and outputs signals from the components of the apparatus 100 can be communicated between the apparatus 100 and an external device (such as a user terminal, or an external command and control system) via a wired communication connection 114.

In FIG. 1a, the apparatus 100 is presented from an end-on perspective. In other words, the multiphase flowable medium would flow in a direction into (or out of) the page. FIG. 1b shows the apparatus 100 viewed from a side perspective, in which the multiphase flowable medium would flow sideways, through a second channel 116b associated with the second sensing location 104b. A first channel associated with the first sensing location 104a is not visible in FIG. 1b, though is substantially similar to the second channel 116b in terms of the features described hereinafter.

The second channel 116b comprises a convergent inlet region 118b, a narrowed region 120b extending from a throat of the convergent inlet region 118b, and a divergent outlet region 122b extending from the narrowed region 120b, opposite the convergent inlet region 118b. In use, it will be understood that flow approaching the apparatus 100, specifically an entrance of either of the channels 116a, 116b of the apparatus will experience an increase in pressure. The convergent inlet region 118b serves only to further increase the pressure. As a result of the tapered profile of the convergent inlet region 118b, a larger area of the oncoming flow is directed through the narrowed region 120b than would be the case without the convergent inlet region 118b, resulting in a more representative sampling of the average local concentration of the substances at the sensing location. The volumetric flow rate of the multiphase flowable medium through the narrowed region 120b is therefore greater than the volumetric flow rate of the multiphase flowable medium through an area with the same cross-section upstream of the apparatus 100. Thus, the increased flow speed through the narrowed region 120b has an additional benefit of reducing the likelihood of blockages of the narrowed region 120b. The divergent outlet region 122b is used to ensure a smooth transition from the flow within the narrowed region 120b back into the steady-state flow within the conduit. In addition, it will be understood that in some examples, the use of convergent regions 118b, 122b on either side of the narrowed region 120b means that the apparatus 100 can be used for flow in either direction.

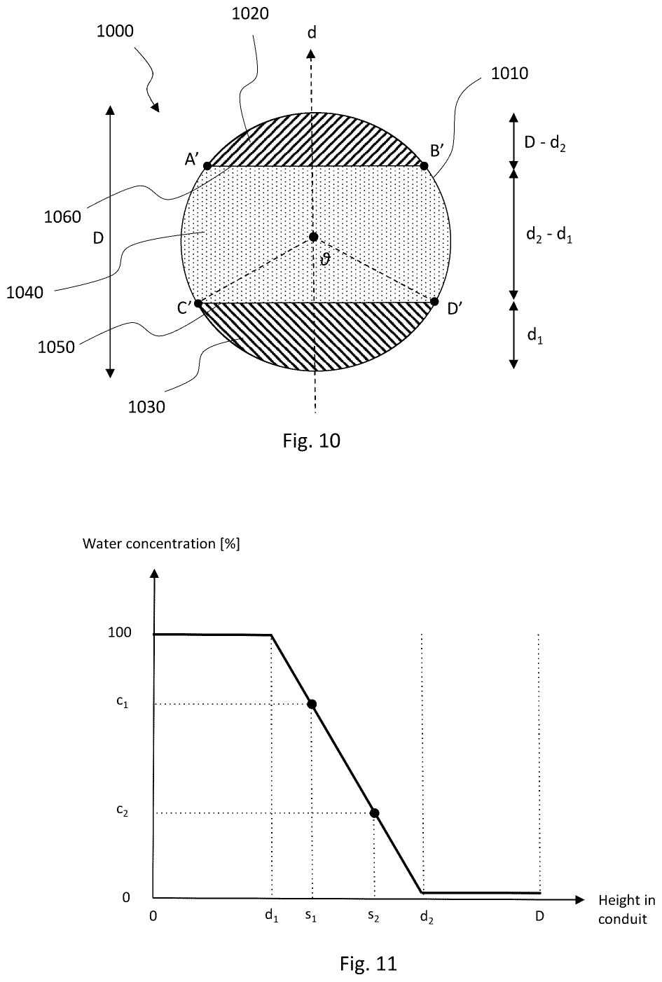

FIG. 2 shows the apparatus of FIGS. 1a and 1b, in use within a conduit. FIG. 2 shows a cross-section through the conduit 200. The features of the apparatus 100 are as described hereinbefore. In FIG. 2, the apparatus 100 is inserted within a conduit 200. The conduit is defined by a first wall 202 and a second wall 204 and has a multiphase flowable medium flowing therethrough. Typically, the conduit 200 has a substantially cylindrical cross-section, such that the first wall 202 and the second wall 204 are simply different regions of the same cylindrical wall. The conduit 200 comprises an upper phase 210, for example of hydrocarbon, a lower phase 220, for example of water, and an intermediate phase 230 formed as an emulsion of the substance of the upper phase 210 and the substance of the lower phase 220. The concentration of each substance in the intermediate phase 230 varies in a known relation (typically substantially linearly) between the boundary with the upper phase 210 and the lower phase 220 (as described further with reference to FIG. 10 hereinafter). The apparatus 100 is inserted into the multiphase flowable medium by insertion through an opening defined in the first wall 202 of the conduit 200. In this way, it can be seen that the probe body 102 of the apparatus 100 extends from the first wall 202 of the conduit 200 towards the second wall 204 of the conduit.

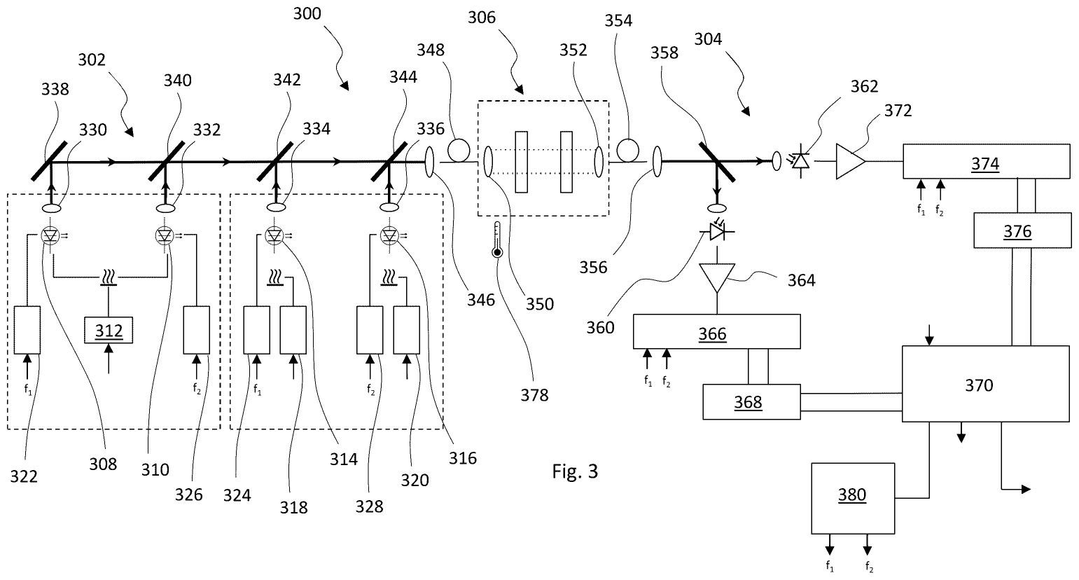

FIG. 3 shows an example of a schematic illustration of an optical and electrical circuit associated with a single sensing location of an apparatus for use in determining one or more parameters of a multiphase flowable medium in accordance with an aspect of the present invention. The circuit 300 includes a source arrangement 302 and a detector arrangement 304, with the sensing location 306, corresponding to the sensing location described with reference to FIGS. 1a, 1b hereinbefore. Specifically, the sensing location 306 is defined in a channel through which the multiphase flowable medium is arranged to flow.

The source arrangement 302 is provided with a plurality of sources configured to emit infrared radiation. In this example, the plurality of sources comprises a first source 308 and a second source 310, each configured to emit infrared radiation for use in determination of one or more parameters indicative of a concentration of hydrocarbon at the sensing location. The first and second sources 308, 310 in this example are in the form of laser diodes, such as Fabry-Perot diodes, having a substantially fixed emission wavelength. In this example, the first source 308 is configured to emit infrared radiation having a wavelength of 1650 nanometres, and a bandwidth of no more than 5 nanometres. The second source 310 is configured to emit infrared radiation having a wavelength of approximately 1725 nanometres and a bandwidth of no more than 5 nanometres. In some examples (not shown in FIG. 3), the wavelength of the infrared radiation emitted from the second source 310 can be adjusted to match the wavelength of the emitted infrared radiation with the peak of the absorption spectrum for the hydrocarbon compound to be analysed, thereby maximising the sensitivity of the resultant detection. The first source 308 and the second source 310 are each configured to be cooled using a first thermoelectric cooler and associated driver 312.

The plurality of sources further comprises a third source 314 and a fourth source 316, each configured to emit infrared radiation for use in determination of one or more parameters indicative of concentration of water at the sensing location. The first and second sources 314, 316 in this example are in the form of lasers, specifically distributed feedback (DFB) lasers, each having a tunable emission wavelength and very narrow emission bandwidth, typically down to only a single wavelength. The third source 314 is configured to emit infrared radiation having a wavelength of approximately 1870 nanometres, but can be varied between approximately 1862 nanometres up to around 1878 nanometres. The fourth source 316 is configured to emit infrared radiation having a wavelength greater than the third source 314, for example approximately 1890 nanometres, but can be varied between approximately 1882 and 1898 nanometres. The third source 314 is configured to be cooled using a second thermoelectric cooler and associated driver 318. The fourth source 316 is configured to be cooled using a third thermoelectric cooler and associated driver 320. It will be understood that the precise wavelength of the emitted infrared radiation is dependent on the temperature of the sources 308, 310, 314, 316. In this way, the use of separate second and third thermoelectric cooler and associated drivers 318, 320 is used to independently control the temperature of the third source 314 and the fourth source 316, allowing the wavelength of the emitted infrared radiation to be tuned as desired. Similarly, by sharing the first thermoelectric cooler and associated driver 312 with both the first source 308 and the second source 310, the wavelengths of infrared radiation emitted by the first and second sources 308, 310 are kept in a matched relationship and therefore are not independently tuned.

It will be understood that the wavelength of the radiation emitted from the first source 308 and the second source 310 is shorter than the wavelength of the radiation emitted from either of the third source 314 or the fourth source 316.

The infrared radiation signal to be emitted from each of the plurality of sources is modulated on a carrier frequency. In this example, the first source 308 and the third source 314 are each modulated at a first frequency f₁, by a respective first source driver 322 and a third source driver 324. The second source 310 and the fourth source 316 are each modulated at a second frequency f₂, by a respective second source driver 326 and a fourth source driver 328.

After emission of the infrared radiation by each of the plurality of sources 308, 310, 314, 316, the emitted radiation is combined and the combined wavelengths are directed towards the sensing location. A plurality of source lenses 330, 332, 334, 336 are provided to direct the emitted radiation towards a respective reflector 338, a first beam combiner 340, a second beam combiner 342 and a third beam combiner 344. A fibre input lens 346 is configured to direct the combined emitted radiation into a single mode fibre optic 348 which optically couples the emitted infrared radiation with the sensing location via a fibre output lens 350. Typically, each of the plurality of sources 308, 310, 314, 316 is operated simultaneously, such that infrared radiation is emitted into the sensing location 306 at all wavelengths of interest at the same time.

Moving to the detector arrangement 304, there is provided a further fibre input lens 352 for directing radiation received at a detector side of the channel at the sensing location into a further fibre optic 354 in the form of a multi-mode fibre optic 354. It will be understood that the multi-mode fibre optic 354 is typically of larger internal cross-sectional area than the single mode fibre optic, and is therefore better arranged to capture relevant infrared radiation from a wider area, for example even where some scattering of the infrared radiation has occurred during the transmission through the multiphase flowable medium. It has been observed that at low concentrations of water, small droplets of water exist in the emulsion, which can act as tiny lenses, redirecting the infrared radiation at the wavelengths of interest. The use of the multi-mode fibre optic 354 ensures that more of this scattered radiation is captured and director onwards towards the photodetectors. An output of the multi-mode fibre optic 354 is directed in a more uniform direction through use of a further fibre output lens 356, from where the received radiation is directed towards a beamsplitter 358, in the form of a longpass beamsplitter 358. Infrared radiation having a wavelength above a predetermined threshold is permitted to pass through the longpass beamsplitter 358. In contrast, infrared radiation having a wavelength below the predetermined threshold is instead reflected by the longpass beamsplitter 358 and does not pass therethrough. In this example, the predetermined threshold is between 1750 nanometres and 1850 nanometres, such as at 1800 nanometres. Accordingly, it will be understood that the infrared radiation received from the first and second sources 308, 310, will be reflected by the longpass beamsplitter 358 towards a first photodetector 360 associated with detection of hydrocarbon-relevant radiation. The infrared radiation received from the third and fourth sources 314, 316 will pass through the longpass beamsplitter 358 to be detected by a second photodetector 362. It will be understood that a photodetector lens may be provided to better direct the received infrared radiation onto each respective photodetector 360, 362.

The signal from the first photodetector 360, having had infrared radiation directed thereto having wavelengths of use in determining one or more parameters indicative of a concentration of hydrocarbons at the sensing location in the multiphase flowable medium, is amplified using a first transimpedance amplifier 364. The first transimpedance amplifier 364 converts the current variations in the signal from the first photodetector 360 into voltage variations, to aid further processing of the signal. Next, the amplified signal is supplied to a first demodulator 366. By demodulating the received signal at the detector, even signals encountering a phase change during transmission through the multiphase flowable medium can be considered. It will be understood that the first demodulator 366 demodulates the input signal from the first transimpedance amplifier 364 into two signals, one each for each of the modulation frequencies f₁, f₂. This will be described more fully with reference to FIG. 5 hereinafter.

A first analogue to digital converter (ADC) 368 is used to convert the analogue voltage output variations of each of the two signals from the first demodulator 366 into two equivalent digital representations as output signals.

The two signals from the first ADC 368 are input to a controller 370 for determining a concentration of the oil at the sensing location, based on the two signals.

Similarly, the output signal from the second photodetector 362, having had infrared radiation directed thereto having wavelengths of use in determining one or more parameters indicative of a concentration of water at the sensing location in the multiphase flowable medium, is amplified using a second transimpedance amplifier 372. The second transimpedance amplifier 372 converts the current variations in the signal from the second photodetector 362 into voltage variations, to aid further processing of the signal. Next, the amplified signal is supplied to a second demodulator 374. By demodulating the received signal at the detector, even signals encountering a phase change during transmission through the multiphase flowable medium can be considered. It will be understood that the second demodulator 374 demodulates the input signal from the second transimpedance amplifier 372 into two signals, one each for each of the modulation frequencies f₁, f₂. This will be described more fully with reference to FIG. 5 hereinafter.

A second analogue to digital converter (ADC) 376 is used to convert the analogue voltage output variations of each of the two signals from the second demodulator 374 into two equivalent digital representations as output signals.

The two signals from the second ADC 376 are input to the controller 370 for determining a concentration of the water at the sensing location, based on the two signals.

The controller 370 also receives as input a temperature signal from a temperature sensor 378, which is arranged to detect the temperature at the sensing location. The temperature signal is used to provide a more accurate value of the concentration of water and oil based on the output signals from the first and second photodetectors 360, 362. The controller 370 is further in data communication with an oscillator component 380 to output the modulation frequencies to be provided to the source drivers 322, 324, 326, 328 and the demodulators 366, 374.

It will be understood that each sensing location of the apparatus is typically provided with a circuit 300 of the type shown in FIG. 3. In other words, apparatus having a plurality of sensing locations typically comprises a corresponding plurality of circuits 300.

Based on the Beer-Lambert law, it will be understood that the intensity of radiation received at a detector, I, can be expressed as:

$$I = {{I}_{o} ( {e}^{- ( {κ}_{w} {c}_{w} + {κ}_{o} {c}_{o} ) x} {e}^{- {κ}_{s} x} )}^{1 - {c}_{g}} {η}_{s y s} + B + N$$

Given that the fractional ratio of water to total fluids, WC, can be expressed as:

$$WC = \frac{{C}_{w}}{{C}_{w} + {C}_{o}}$$

and that the fractional ratio of gas to the sum of all flow constituents, GVF, can be expressed as: GVF=c_{g}

The preceding equation for I can be expressed in terms of WC and GVF and simplified as:

$$I = {I}_{o} {e}^{- ( {κ}_{w} WC + {κ}_{o} ( 1 - WC ) + {κ}_{s} ) ( 1 - GVF ) x} {η}_{sys} + B + N$$

where:
I and Iₒ are the received and source intensity respectively
k_{w} and kₒ are the resonant absorption coefficients for water and oil respectively
kₛ is the non-resonant scattering coefficient
c_{w}, cₒ, c_{g} are the fractional concentrations of water, hydrocarbon and gas respectively
x is the normal interaction length with the flow
n_{sys} is the optical efficiency of the system
B is the broadband background blackbody radiation
N is the noise in the contribution of electronic noise in the system

The disclosed apparatus includes two measurements for water having wavelengths which are only slightly spaced apart. For hydrocarbon detection, similarly two measurements are used.

It will be understood that the first and second demodulators 366, 374 described above, and described further with reference to FIG. 9 hereinafter, can be used to significantly suppress the contribution from the broadband background blackbody radiation, B, and the noise in the contribution of electronic noise in the system, N. Furthermore, where two measurements are taken at closely spaced wavelengths then the optical efficiency of the system and the non-resonant scattering coefficient can be considered substantially similar at both wavelengths. By careful choice of particular wavelength regions, it is possible to pick two wavelengths at which the resonant absorption coefficients for one substance (e.g. water) would be different, whilst the resonant absorption coefficients for another substance (e.g. hydrocarbon) would be very similar.

By determining a ratio of each pair of measurements related to a single substance (water or hydrocarbon), the ratio of I(λ₁):I(λ₂) can be considered, in a simplified form, to be:

$$\frac{I ( {λ}_{1} )}{I ( {λ}_{2} )} = \frac{{e}^{- ( {κ}_{w} ( {λ}_{1} ) WC + {κ}_{o} ( {λ}_{1} ) ( 1 - WC ) ) ( 1 - GVF ) x}}{{e}^{- ( {κ}_{w} ( {λ}_{2} ) WC + {κ}_{o} ( {λ}_{2} ) ( 1 - WC ) ) ( 1 - GVF ) x}}$$

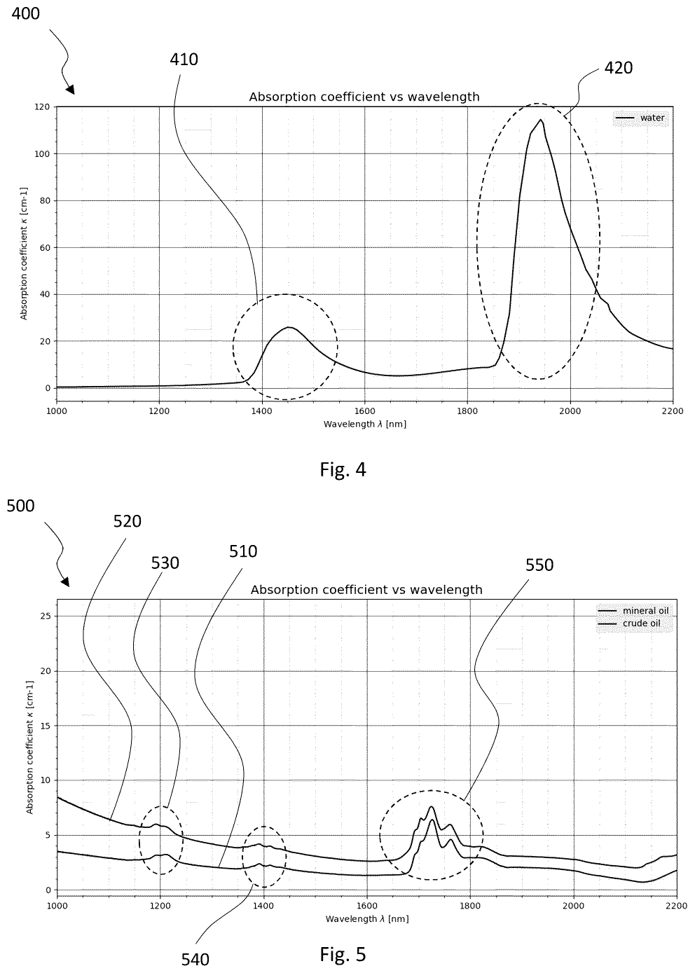

As described hereinbefore, the two measurements for water are done at very close wavelengths. At this point, it is thought helpful to discuss the absorption spectrum for water and for oil, and the related infrared radiation measurements to be conducted by the apparatus described herein. FIG. 4 shows an absorption spectrum of water. Specifically, the absorption spectrum 400 is shown within the wavelength range of 1000 nanometres to 2200 nanometres. It can be seen that there exists a first absorption peak 410 having a locally-maximum absorption coefficient of around 26 cm⁻¹ at around 1450 nanometres, and extending between around 1375 nanometres to around 1600 nanometres. There is also a second absorption peak 420 having a locally-maximum absorption coefficient of around 115 cm⁻¹ at 1940 nanometres, and extending between around 1850 nanometres to around 2200 nanometres.

As will be appreciated, the second absorption peak 420 has a much greater response that the first absorption peak 410, the second absorption peak 420 also having a far steeper gradient that the first absorption peak 410, particularly in the region of the second absorption peak 420 below the associated locally-maximum absorption coefficient of the second absorption peak 420.

FIG. 5 shows absorption spectra of two different hydrocarbons. The absorption spectra 500 includes an absorption spectrum for mineral oil 510 and an absorption spectrum for crude oil 520. Hydrocarbons (such as mineral oil and crude oil) include a number of absorption features in the wavelength range between 1000 nanometres and 2200 nanometres. Specifically, it can be seen that a first absorption peak 530 exists at around 1200 nanometres, a second absorption peak 540 exists around 1400 nanometres and a third absorption peak 550 exists around 1725 nanometres. It can be seen that the third absorption peak 550 is more pronounced than either of the first absorption peak 530 or the second absorption peak 540. The third absorption peak has a locally-maximum absorption coefficient of around 7.5 cm⁻¹ at 1725 nanometres, and extends between around 1675 nanometres and 1780 nanometres.

It will be noted that FIGS. 4 and 5 are shown across the same range of wavelengths, allowing easy comparison to be made between the spectrums for water and hydrocarbons. The present inventors have realised that at the second absorption peak 420 of water, there is only minimal change in the absorption coefficient for oil, particularly in the region of the second absorption peak 420 having a wavelength less than the locally-maximum absorption coefficient between 1875 nanometres and 1950 nanometres. Similarly, at the third absorption peak 550 of hydrocarbon absorption spectra 510, 520, there is only minimal change in the absorption coefficient for water between 1600 nanometres and 1750 nanometres. Accordingly, the present inventors have further realised that it is possible to discount the contribution of either oil or water when measuring the variation in the absorption by the other of oil and water at particular wavelengths, where two measurement wavelengths are used which are closely spaced, and where the ratio of the responses are determined.

Specifically, for measuring the concentration of water, by computing the ratio of responses at two different wavelengths, as in the formular provided hereinbefore, those two wavelengths are typically within the range of between 1860 and around 1950, meaning that the variation in the contribution from oil is substantially negligible when the ratio is determined. Furthermore, where the two measurement wavelengths are sufficiently close, such as within 50 nanometres, the contribution from the variation in the non-resonant scattering coefficient is also substantially negligible in the ratio of the responses.

In other words, the ratio of the responses for water can be simplified as:

$$\frac{I ( {λ}_{1} )}{I ( {λ}_{2} )} = \frac{{e}^{- ( {κ}_{w} ( {λ}_{1} ) WC ) ( 1 - GVF ) x}}{{e}^{- ( {κ}_{w} ( {λ}_{2} ) WC ) ( 1 - GVF ) x}} = {e}^{( {κ}_{w} ( {λ}_{2} ) - {κ}_{w} ( {λ}_{1} ) ) WC ( 1 - GVF ) x}$$

which can be solved for the concentration of water, WC.

Similarly, for measuring the concentration of oil, by computing the ratio of responses at two different wavelengths, as in the formular provided hereinbefore, those two wavelengths are typically within the range of between 1650 and around 1740, meaning that the variation in the contribution from water is substantially negligible. Furthermore, where the two measurement wavelengths are sufficiently close, the contribution from the variation in the non-resonant scattering coefficient is also substantially negligible in the ratio of the responses.

In other words, the ratio of the responses for hydrocarbons can be simplified as:

$$\frac{I ( {λ}_{1} )}{I ( {λ}_{2} )} = \frac{{e}^{- ( {κ}_{o} ( {λ}_{1} ) ( 1 - WC ) ) ( 1 - GVF ) x}}{{e}^{- ( {κ}_{o} ( {λ}_{2} ) ( 1 - WC ) ) ( 1 - GVF ) x}} = {e}^{( {κ}_{o} ( {λ}_{2} ) - {κ}_{o} ( {λ}_{1} ) ) ( 1 - WC ) ( 1 - GVF ) x}$$

which can also be solved for the fractional ratio of hydrocarbon to total fluids (1−WC). It will further be understood that by taking the ratio between the ratio of the responses for water and the ratio of the responses for hydrocarbons, the (1−GVF) terms cancel each other out, allowing the equation to be solved for the fractional ratio of water to total fluids, WC.

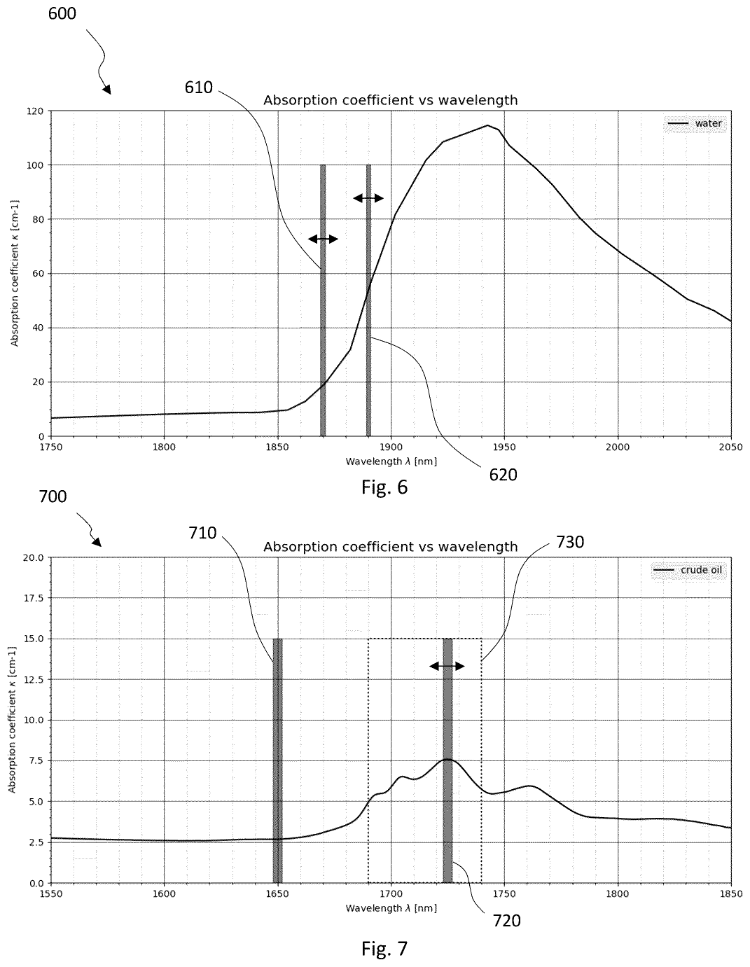

FIG. 6 shows measurement bands relative to the absorption spectrum of water 600 in accordance with an aspect of the present invention. As can be seen, there is provided a first measurement band 610 around 1870 nanometres and a second measurement band 620 around 1890 nanometres. Thus, the two measurements bands 610, 620 are spaced by only 20 nanometres from each other. The first measurement band 610 can be tuned slightly to a higher or lower wavelength as desired. Similarly, the second measurement band 620 can also be tuned slightly to a higher or lower wavelength as desired. In this way, the accuracy of the resulting concentration determination can be improved by moving the measurement bands more closely together, thereby resulting in an even smaller variation in the coefficients of absorption of oil and the non-resonant scattering coefficient between the two measurement bands 610, 620. However, it will be understood that particularly closely-spaced measurement bands 610, 620 may reduce the sensitivity of the detection, and so may not be suited for use with multiphase flowable mediums having only very low concentrations of water. At other times, the sensitivity of the resulting concentration determination can be improved by spacing the measurement bands 610, 620 slightly further apart to ensure a larger difference between the absorption coefficients of water at the two wavelengths. However, it will be understood that increasing the spacing between the measurement bands 610, 620 may reduce accuracy of the concentration determination because there will be a greater variation in the absorption coefficient of oil, and in the non-resonant scattering coefficient between the two measurement bands 610, 620. The first measurement band 610 and the second measurement band 620 each have a narrow bandwidth, in this example substantially a single wavelength.

FIG. 7 shows measurement bands relative to the absorption spectrum of crude oil 700 in accordance with an aspect of the present invention. Similarly to water, there is provided a first measurement band 710 around 1650 nanometres, and a second measurement band 720 shown at around 1725 nanometres. Depending on the particular hydrocarbon compound to be identified and/or the operating parameters of the application, the second measurement band 720 may be selected to be anywhere within a wavelength range 730 from around 1690 nanometres to around 1740 nanometres. The first measurement band 710 has a substantially narrow bandwidth, for example no more than 5 nanometres. The second measurement band 720 also has a substantially narrow bandwidth, for example no more than 5 nanometres.

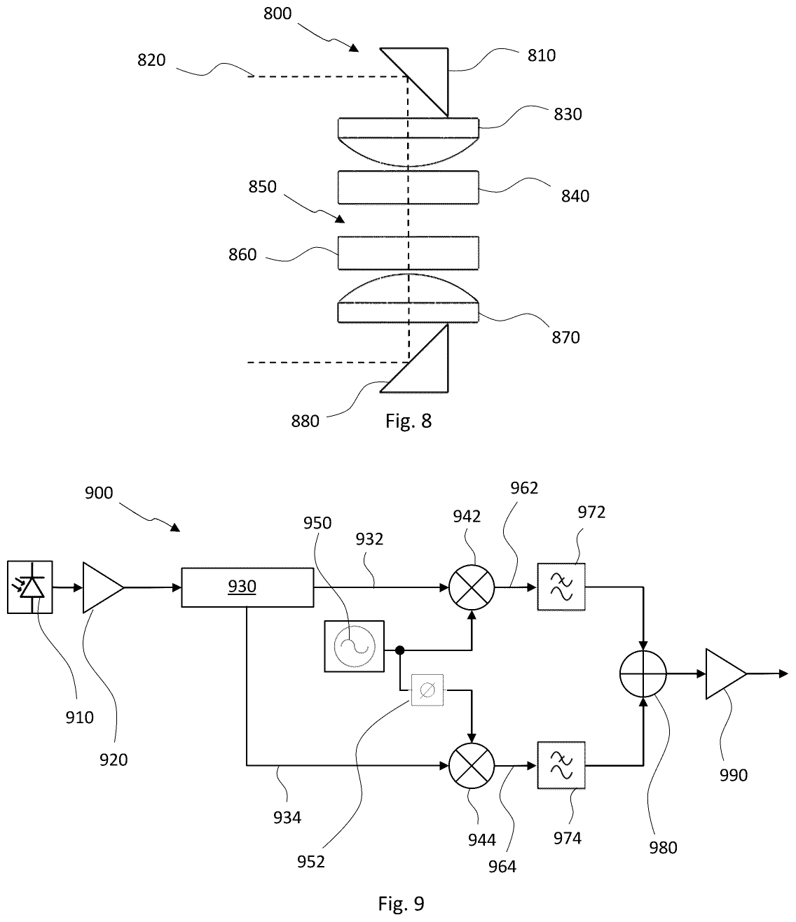

FIG. 8 is a graphical illustration of an optical arrangement for use in the circuit of FIG. 3. The optical arrangement 800 covers the region of the sensing location 306 shown in FIG. 3. Specifically, the optical arrangement 800 comprises a first reflector 810, in the form of a prism, for reflecting a source ray of infrared radiation 820 by substantially 90 degrees, through a source lens 830, a source channel window 840, through the channel 850 through which the multiphase flowable medium is arranged to flow, in use, onwards from the channel 850 through a detector channel window 860, through a detector lens 870, and to be reflected by a second reflector 880, in the form of a prism, to be directed towards the photodetectors (not shown in FIG. 8).

FIG. 9 shows a schematic illustration of a further circuit for use in the circuit 300 of FIG. 3. The further circuit shown in FIG. 9 is a demodulation circuit 900, and illustrates the functionality of part of the first and second demodulators 366, 374. For clarity, the functionality of the demodulation circuit 900 of FIG. 9 will be described with reference to the signal received from the second photodetector 362, associated with the detection of wavelengths relevant for determination of a concentration of water in the multiphase flowable medium. Specifically, the output from a photodetector 910 (e.g. the second photodetector 362 of FIG. 3) is amplified by a transimpedance amplifier 920 (e.g. the second transimpedance amplifier 372 of FIG. 3) to convert the variations in current in the signal output from the photodetector 910 into variations in voltage of the signal output from the transimpedance amplifier 920. The signal output from the transimpedance amplifier 920 is input to a bandpass filter 930 to isolate signals at the specific modulation frequency. It will be appreciated that whilst all of the signal from the photodetector 910 can be processed by a single transimpedance amplifier 920, each wavelength of interest (and therefore modulation frequency) will require a separate bandpass filter, with the signal being split between each of the bandpass filters of any corresponding demodulation circuits 900 to use the signal from the same photodetector 910. In the present example, there are two wavelengths of interest to be detected by both the first photodetector 360 and the second photodetector 362 of FIG. 3, meaning that the system includes four copies of the demodulation circuit 900 (excluding the photodetector 910 and the transimpedance amplifier 920, which are shared between two copies of the rest of the demodulation circuit 900). The output from the bandpass filter 930 is fed as two identical signals 932, 934, into a first demodulator 942 a second demodulator 944 respectively. The first and second demodulators 942, 944 are controlled to demodulate the first and second signals 932, 934 according to a demodulation frequency provided from a frequency input signal 950. The demodulation frequency is the same as the modulation frequency at which the emitted infrared radiation was modulated, and therefore is indicative of the wavelength of interest. The demodulation frequency signal supplied to the second demodulator 944 is phase shifted to be 90 degrees out of phase with the demodulation frequency signal supplied to the first demodulator 942, by a phase shifter 952. Accordingly, a first demodulated signal 962 from the first demodulator 942 comprises parts of the original signal not present in a second demodulated signal 964 from the second demodulator 944. The first and second demodulated signals 962, 964 are next input to respective first and second low pass filters 972, 974 to remove high frequency noise components therefrom, before being combined in a signal combiner 980. The combined signal is further amplified using a DC amplifier 990 to provide the signal indicative of the intensity of light received at the photodetector corresponding to the wavelength of interest. Specifically, the signal is proportional to, if not corresponds to, I(λ₁) or I(λ₂) from the equations described hereinbefore.

The circuit 900 described with reference to FIG. 9 can sometimes be referred to as a lock-in amplifier, and is used to suppress the blackbody background radiation, measurement noise and DC offsets.

Thus, it will be understood that the apparatus and circuits described hereinbefore can be used to determine a concentration of a substance, for example water and/or a hydrocarbon, at one or more sensing locations within a multiphase flowable medium.

The quantification of the absolute amount of water and oil in the conduit can be further enhanced by exploiting the fact that any gas does not typically have a significant contribution to the absorption of electromagnetic radiation caused by the multiphase flowable medium, whereas liquid water and oil do. Furthermore, gas is a compressible fluid whereas water and hydrocarbons can each be considered substantially incompressible, meaning that any change in the concentration of gas in the conduit does not lead to a change in the volume occupied by the water or the hydrocarbon, only to a change in the pressure within the conduit As the sensing volume is finite, an increasing amount of one liquid must mean a relatively decreasing amount of the other liquid. By taking the ratio of one liquid to the other, it is possible to further enhance the response to changing concentrations of liquids as well as the robustness of absolute concentration measurements. The latter is particularly important when the gas concentration is high i.e. the water and hydrocarbon signals are weak.

The present inventors further realised that by calculating the concentration of a substance at two spaced locations through a cross-section of the multiphase flowable medium, the two spaced locations both within an emulsion phase of the multiphase flowable medium, an estimate of the concentration gradient of the emulsion phase could be determined, and from that, the phase boundaries between three phases in the multiphase flowable medium could further be determined.

FIG. 10 shows a schematic diagram of a conduit, illustrating an example phase arrangement within a multiphase flowable medium flowing therethrough. The schematic diagram 1000 shows the boundary of the conduit 1010. The conduit 1010 comprises: a first phase 1020, in this example a hydrocarbon phase 1020 at an upper region of the conduit 1010; a second phase 1030, in this example a water phase 1030 at a lower region of the conduit 1010; and an intermediate phase 1040, in this example an emulsion phase 1040 being an emulsion of the water and the hydrocarbon, extending between the hydrocarbon phase 1020 and the water phase 1030 in a central region of the conduit 1010. A concentration of water in the emulsion phase 1040 varies substantially linearly with distance through the emulsion phase 1040 from substantially 100% water at a water boundary 1050 of the emulsion phase 1040 and the water phase 1030, to substantially 0% water at a hydrocarbon boundary 1060 of the emulsion phase 1040 and the hydrocarbon phase 1020. Similarly, a concentration of hydrocarbon in the emulsion phase 1040 varies substantially linearly with distance through the emulsion phase 1040 from substantially 0% hydrocarbon at a water boundary 1050 of the emulsion phase 1040 and the water phase 1030, to substantially 100% hydrocarbon at a hydrocarbon boundary 1060 of the emulsion phase 1040 and the hydrocarbon phase 1020. It will be understood that from the water boundary 1050 to the lower surface of the conduit 1010, within the water phase 1030, the concentration of water is substantially 100%, and the concentration of hydrocarbon is substantially 0%. Similarly, from the hydrocarbon boundary 1060 to the upper surface of the conduit 1010, within the hydrocarbon phase 1020, the concentration of water is substantially 0% and the concentration of hydrocarbon is substantially 100%.

FIG. 11 is a graph showing a concentration of water (y-axis) with the distance through a cross-section of the conduit in the d direction, for the phase arrangement of FIG. 10. Specifically, by measuring a first percentage concentration, c₁, of water in the emulsion phase 1040 at a first known sensing location, s₁, and measuring a second percentage concentration, c₂, of water in the emulsion phase 1040 at a second known sensing location, s₂, the concentration gradient in the emulsion can be calculated as:

$$m = \frac{Δ c}{Δ d} = \frac{{c}_{1} - {c}_{2}}{{s}_{1} - {s}_{2}}$$

Assuming that the first sensing location, s₁, and the second sensing location, s₂, are both within the emulsion phase 1040, then the boundaries between the emulsion phase 1040 and either of the other two adjacent phases can be determined by calculating the position at which the concentration of water is 0%, and the position at which the concentration of water is 100%. It will be understood that the concentration at location d (within the emulsion phase 1040), c(d), is:

$$c ( d ) = {c}_{1} + ( d - {s}_{1} ) \frac{( {c}_{1} - {c}_{2} )}{( {s}_{1} - {s}_{2} )}$$

Accordingly, the boundary between the water phase 1030 and the emulsion phase 1040, d₁, is:

$${d}_{1} = \frac{( 100 - {c}_{1} ) ( {s}_{1} - {s}_{2} )}{( {c}_{1} - {c}_{2} )} + {s}_{1}$$

The boundary between the hydrocarbon phase 1020 and the emulsion phase 1040, d₂, is:

$${d}_{2} = \frac{( {c}_{1} ) ( {s}_{2} - {s}_{1} )}{( {c}_{1} - {c}_{2} )} + {s}_{1}$$

Thus, it will be understood that the average percentage water concentration, c̅, in the emulsion can therefore be determined as:

$$\overline{c} = c ( \frac{{d}_{2} - {d}_{1}}{2} )$$

Furthermore, a degree of emulsion stratification, S, can be determined as:

$$S = ( \frac{{tan}^{- 1} ( \vert m \vert )}{90 °} ) 100 \%$$

Although it is shown in FIGS. 10 and 11 that the two sensing locations are provided within the emulsion phase 1040, this is not guaranteed and it may be that one or both of the sensing locations s₁, s₂, are located in a phase other than the emulsion phase 1040. For example, it may be that the first sensing location s₁ is positioned within the water phase 1030 and/or that the second sensing location s₂ is positioned within the hydrocarbon phase 1020.

Where the concentrations c₁ and c₂ are each both greater than 0% and less than 100%, then it is clear that both sensing locations s₁, s₂, are located in the emulsion phase 1040, with the water phase thickness being d₁, the hydrocarbon phase thickness being D−d₂ (D being the diameter of the pipe), and the emulsion phase thickness being d₂−d₁.

However, where any concentration c₁, c₂ is around 0% or around 100%, it is clear that at least one of the sensing locations is outside the emulsion phase 1040.

Furthermore, if d₂ is determined to be less than D or the concentration c₂ (concentration of water at the second sensing location) is approximately 0%, it is clear that a hydrocarbon phase must exist in the conduit. If concentration c₂ is not 0%, then the hydrocarbon layer boundary must be between s₂ and D, and the hydrocarbon layer thickness is D−d₂, as above. Alternatively (where concentration c₂ is approximately 0%), if the concentration c₁ is not 0%, then the hydrocarbon layer boundary must be between s₁ and s₂. It is not possible to calculate a precise location of the hydrocarbon layer boundary, so the hydrocarbon layer boundary can be estimated to be at the midpoint between s₁ and s₂. In other words, the hydrocarbon layer thickness can be D−((s₂−s₁)/2). Finally, if, instead, c₂ and c₁ are both 0%, it is clear that the hydrocarbon layer boundary must be between s₁ and the edge of the conduit below s₁. The hydrocarbon layer thickness can be estimated no more precisely than being at D−s₁.

If neither d₂ is less than D, nor c₂ is around 0%, then it is likely that no hydrocarbon phase is present at all. There may still be a water phase present, however. If d₁ is greater than 0 and/or c₁ is approximately 100%, this indicates that a water phase exists in the conduit. If concentration c₁ is not 100%, then the water phase boundary is between the edge of the conduit and the first sensing location s₁. The water phase thickness can be estimated as d₁. If the concentration c₂, being the water concentration at the second sensing location, is not 100%, then the water phase boundary is between s₁ and s₂. The water phase thickness can be estimated no more precisely than being from the wall of the conduit to the midpoint between the sensing locations s₁, s₂. Specifically, the water phase thickness can be estimated as (s₂−s₁)/2. Alternatively, if the concentrations c₁, c₂ are both 100%, the water phase boundary must be beyond $2, and the water phase thickness can be estimated no more precisely than being at s₂.

The example above is one set of logic for making a determination of a probable distribution of fluid phases within the conduit based on measurements of concentrations from two known locations within a cross-section through the conduit.

Given the estimate of the thicknesses and locations of each of the phases of water and hydrocarbon and the emulsion phase, the percentage water content in the conduit can be determined as the average concentration of water in the conduit, c̅_{conduit}.

It will be understood that the percentage water content in the conduit can be expressed as:

$${\overline{c}}_{conduit} = \frac{{\overline{c}}_{oil} {A}_{oil} + {\overline{c}}_{emulsion} {A}_{emulsion} + {\overline{c}}_{water} {A}_{water}}{{A}_{conduit}} 100 \%$$

### Where:

A_{conduit} is the cross-sectional area of the conduit;
c̅ₒᵢₗ is the average percentage concentration of water in the hydrocarbon phase;
Aₒᵢₗ is the cross-sectional area of the hydrocarbon phase;
c̅ₑₘᵤₗₛᵢₒₙ is the average percentage concentration of water in the emulsion phase;
Aₑₘᵤₗₛᵢₒₙ is the cross-sectional area of the emulsion phase;
c̅_{water} is the average percentage concentration of water in the water phase; and
A_{water} is the cross-sectional area of the water phase.

Given that the concentration of water in the hydrocarbon phase is 0% and the concentration of water in the water phase is 100%, this can be re-written as:

$${\overline{c}}_{conduit} = \frac{{\overline{c}}_{emulsion} {A}_{emulsion} + {A}_{water}}{{A}_{conduit}} 100 \%$$

Returning to FIG. 10, it will be understood that for a conduit having a circular cross-section, the area of the water phase 1030 can be calculated as:

$${A}_{water} = ( \frac{{θ}_{{C}^{′} - {D}^{′}} π}{360} - \frac{sin {θ}_{{C}^{′} - {D}^{′}}}{2} ) {( \frac{D}{2} )}^{2}$$

The area of the emulsion can be calculated as:

$${A}_{emulsion} = ( \frac{{θ}_{{A}^{′} - {B}^{′}} π}{360} - \frac{sin {θ}_{{A}^{′} - {B}^{′}}}{2} ) {( \frac{D}{2} )}^{2} - {A}_{water}$$

Thus, it will be understood that the percentage water content of the multiphase flowable medium in the conduit can be determined. The percentage hydrocarbon content can be determined with appropriate changes to the formulas and methods described hereinbefore.

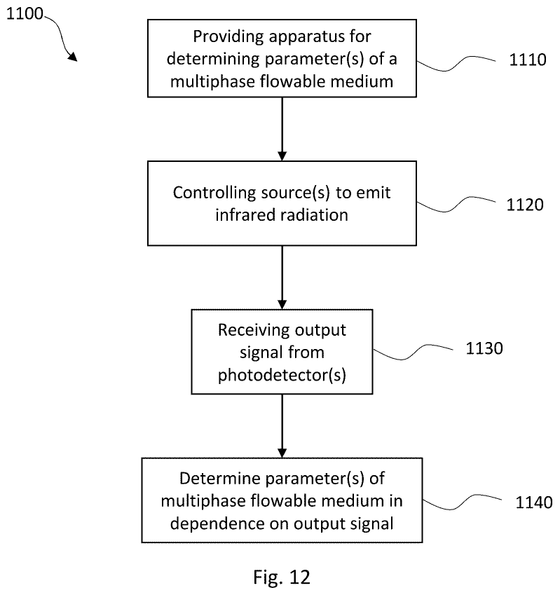

FIGS. 12 to 14 are flow diagrams illustrating methods of determining parameters of the multiphase flowable medium in accordance with aspects of the present inventions.

FIG. 12 shows a method 1100 of determining one or more parameters of a multiphase flowable medium flowing in a flow direction through a conduit, substantially as described hereinbefore, wherein the method 1100 comprises determining the one or more parameters in dependence on an output signal from a photodetector at each of a plurality of sensing locations in a cross-section through the conduit. Specifically, the method 1100 comprises providing 1110 the apparatus as described hereinbefore. Subsequently, the method 1100 comprises controlling 1120 at least one source of each sensing location to emit infrared radiation into the multiphase flowable medium flowing through the conduit at the respective sensing location. The method 1100 further comprises receiving 1130 an output signal from at least one photodetector of each sensing location. The output signal is indicative of the infrared radiation from the at least one source, received at the at least one photodetector, having passed through the multiphase flowable medium in the conduit at the sensing location. The method 1100 further comprises determining 1140 one or more parameters of the multiphase flowable medium in dependence on the received output signal. It may be that the method 1100 further comprises outputting the determined one or more parameters, such as via an electronic communication unit.

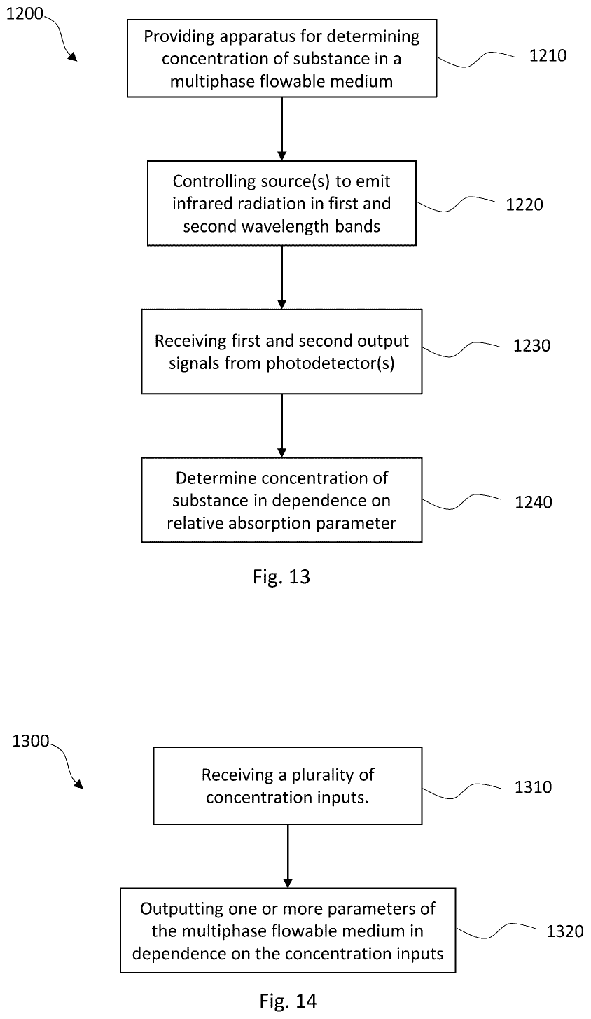

FIG. 13 shows a method 1200 of determining an output indicative of a concentration of a substance in a multiphase flowable medium flowing in a flow direction through a conduit. In a broad sense, the method 1200 comprises determining the output indicative of the concentration of the substance in the multiphase flowable medium in dependence on a relative absorption parameters determined based on infrared radiation received in a first wavelength band and in a second wavelength band. Specifically, the method 1200 comprises providing 1210 the apparatus as described hereinbefore. Subsequently, the method 1200 comprises controlling 1220 at least one source to emit infrared radiation into the multiphase flowable medium flowing through the conduit, the infrared radiation emitted in the first wavelength band and in the second wavelength band. The method 1200 further comprises receiving 1230, from the at least one photodetector, a first output signal indicative of infrared radiation received in the first wavelength band, and a second output signal indicative of infrared radiation received in the second wavelength band. The method 1200 further comprises determining 1240 the output indicative of the concentration of the substance in the multiphase flowable medium, based on a relative absorption parameter. The relative absorption parameter is determined in dependence on the first output signal and the second output signal.

FIG. 14 shows a method of determining one or more parameters of a multiphase flowable medium flowing in a flow direction through a conduit. Broadly, the method 1300 is a method of determining the one or more parameters in dependence on a plurality of concentration inputs, each as a mutually spaced position in a direction transverse to the flow direction. Specifically, the method 1300 comprises receiving 1310 a plurality of concentration inputs. Each concentration input is indicative of a concentration of at least one substance of the multiphase flowable medium in the conduit at a respective position within the conduit. Each position is typically mutually spaced in a direction transverse to the flow direction. The method 1300 further comprises outputting 1320 one or more parameters of the multiphase flowable medium in dependence on the plurality of concentration inputs.

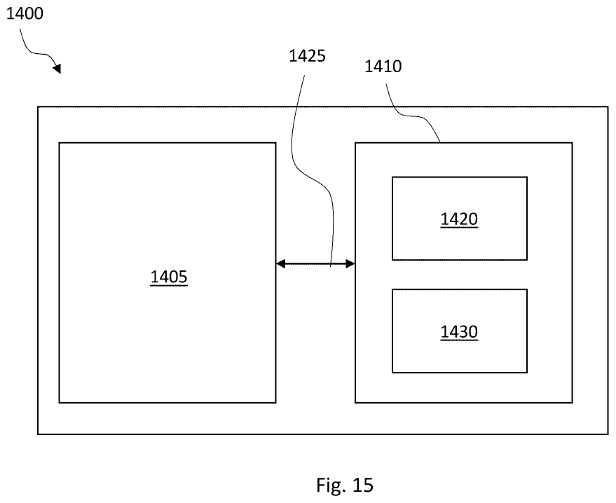

FIG. 15 is a schematic illustration of an apparatus comprising a controller in accordance with an aspect of the present invention. The apparatus 1400 comprises a controller 1410 for exchanging data and/or control signals 1425 with one or more further components 1405 of the apparatus 1400, such as the sources and/or photodetectors described hereinbefore. The controller 1410 comprises one or more processors 1420 and a computer-readable non-transient memory 1430. The computer-readable non-transient memory 1430 stores instructions which, when executed by the one or more processors 1420, causes operation of the components 1405 of the apparatus 1400 in accordance with the methods hereinbefore described.

As described hereinbefore, it will be understood that in some examples the one or more processors 1420 may be located in a single unit. In other examples, where the one or more processors 1420 is a plurality of processors, the controller 1410 may be distributed, which is to say that at least one of the plurality of processors 1420 may be located separated from at least one other of the plurality of processors 1420.

In summary, there is provided apparatus (100) for use in determining one or more parameters of a multiphase flowable medium flowing in a flow direction through a conduit (200). The multiphase flowable medium comprises at least a water phase (220). The apparatus (100) comprises: a probe body (102) for extending from a wall (202) of a conduit (200), into a multiphase flowable medium flowing therethrough. The probe body (102) defines a plurality of sensing locations (104a, 104b), each for a different portion of the multiphase flowable medium and mutually spaced from any other one of the plurality of sensing locations (104a, 104b) in a direction having at least a component transverse to the flow direction. Each sensing location (104a, 104b) is provided with: at least one source (308, 310, 314, 316) configured to emit infrared radiation into the multiphase flowable medium; and at least one photodetector (360, 362) configured to detect infrared radiation received from the at least one source via the multiphase flowable medium.

Throughout the description and claims of this specification, the words “comprise” and “contain” and variations of them mean “including but not limited to”, and they are not intended to and do not exclude other components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, or groups described in conjunction with a particular aspect, embodiment, or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. Apparatus for use in determining one or more parameters of a multiphase flowable medium flowing in a flow direction through a conduit, the multiphase flowable medium comprising at least a water phase, the apparatus comprising:
a probe body for extending from a wall of a conduit, into a multiphase flowable medium flowing therethrough, the probe body defining a plurality of sensing locations, each for a different portion of the multiphase flowable medium and mutually spaced from any other one of the plurality of sensing locations in a direction having at least a component transverse to the flow direction,
wherein each sensing location is provided with:
at least one source configured to emit radiation from along an infrared spectrum into the multiphase flowable medium; and
at least one photodetector configured to detect infrared radiation received from the at least one source via the multiphase flowable medium.

2. The apparatus of claim 1, wherein:
the at least one source of each sensing location is configured to emit infrared radiation in a first water wavelength band, and in a second water wavelength band,
each of the first water wavelength band and the second water wavelength band being a region of the infrared spectrum in which the water phase exhibits absorption; and
the at least one source of each sensing location is configured to emit a reduced amount of infrared radiation in an intermediate wavelength band between the first water wavelength band and the second water wavelength band.

3. The apparatus of claim 1, wherein:
the at least one photodetector of each sensing location is configured to detect infrared radiation in the or a first water wavelength band, or the second water wavelength band; and
each of the first water wavelength band and the second water wavelength band resides within a region of the infrared spectrum in which the water phase exhibits absorption.

4. The apparatus of claim 2, wherein the first water wavelength band and the second water wavelength band each include one or more wavelengths between 1850 nanometres and 1950 nanometres.

5. The apparatus of claim 2, wherein:
the first water wavelength band and the second water wavelength band each have a 3 dB bandwidth of less than 10 nanometres, and/or
the first water wavelength band is separated from the second water wavelength band by less than 50 nanometres.

6. The apparatus of claim 2, wherein the apparatus is configured to adjust a centre wavelength of the first water wavelength band within a first water wavelength range, and to adjust a centre wavelength of the second water wavelength band within a second water wavelength range.

7. The apparatus of claim 1, wherein:
the multiphase flowable medium further comprises a hydrocarbon phase,
the at least one source of each sensing location is configured to emit infrared radiation in a first hydrocarbon wavelength band, and in a second hydrocarbon wavelength band, and
at least one of the first hydrocarbon wavelength band and the second hydrocarbon wavelength band resides within a region of the infrared spectrum in which the hydrocarbon phase exhibits absorption.

8. The apparatus of claim 1, wherein:
the multiphase flowable medium further comprises a hydrocarbon phase,
the at least one photodetector of each sensing location is configured to detect infrared radiation in the first hydrocarbon wavelength band or in the second hydrocarbon wavelength band, and
at least one of the first hydrocarbon wavelength band and the second hydrocarbon wavelength band resides within a region of the infrared spectrum in which the hydrocarbon phase exhibits absorption.

9. The apparatus of claim 7, wherein the first hydrocarbon wavelength band and the second hydrocarbon wavelength band each include one or more wavelengths between 1620 nanometres and 1750 nanometres.

10. The apparatus of claim 7, wherein:
the first hydrocarbon wavelength band and the second hydrocarbon wavelength band each have a 3 dB bandwidth of less than 10 nanometres, and
the first hydrocarbon wavelength band is separated from the second hydrocarbon wavelength band by more than 30 nanometres.

11. The apparatus of claim 2, wherein;
the multiphase flowable medium further comprises a hydrocarbon phase;
the at least one source of each sensing location is configured to emit infrared radiation in a first hydrocarbon wavelength band, and in a second hydrocarbon wavelength band;
at least one of the first hydrocarbon wavelength band and the second hydrocarbon wavelength band resides within a region of the infrared spectrum in which the hydrocarbon phase exhibits absorption;
the at least one source comprises:
a first source configured to emit infrared radiation in substantially only the first water wavelength band;
a second source configured to emit infrared radiation in substantially only the second water wavelength band;
a third source configured to emit infrared radiation in substantially only the first hydrocarbon wavelength band; and
a fourth source configured to emit infrared radiation in substantially only the second hydrocarbon wavelength band, and
the at least one photodetector comprises:
a first photodetector configured to detect infrared radiation in the first water wavelength band and the second water wavelength band; and
a second photodetector configured to detect infrared radiation in the first hydrocarbon wavelength band and the second hydrocarbon wavelength band.

12. The apparatus of claim 11, wherein each sensing location is further provided with:
a source optical arrangement configured to combine the infrared radiation emitted from each of the first source, the second source, the third source and the fourth source to be directed together through the multiphase flowable medium at the sensing location, and
a detector optical arrangement configured to split the infrared radiation received from the multiphase flowable medium into a first wavelength band to be directed to the first photodetector and a second wavelength band to be directed to the second photodetector.

13. The apparatus of claim 1, wherein a minimum width of a channel defined by the probe body, and through which infrared radiation from the at least one source must pass to be detected by the at least one photodetector is less than five millimetres.

14. The apparatus of claim 1, wherein the probe body defines a plurality of channels, with each channel of the plurality of channels being associated with a respective sensing location of the plurality of sensing locations.

15. The apparatus of claim 1, wherein the probe body defines at least one channel, with each channel of the at least one channel being provided with a convergent region forming a mouth of the respective channel.

16. The apparatus of claim 1, further comprising:
a controller configured to determine an output indicative of a concentration of water in the multiphase flowable medium at each sensing location, in response to an output signal from the at least one photodetector for the respective sensing location.

17. The apparatus claim 4, further comprising:
a controller configured to determine an output indicative of a concentration of water in the multiphase flowable medium at each sensing location in response to an output signal from the at least one photodetector for the respective sensing location, and
wherein the controller is configured to determine the output indicative of the concentration of water in dependence on determining a relative water response parameter in dependence on a first output signal from the at least one photodetector, indicative of infrared radiation received in the first water wavelength band, and a second output signal from the at least one photodetector, indicative of infrared radiation in the second water wavelength band.

18. The apparatus of claim 17, wherein;
the multiphase flowable medium further comprises a hydrocarbon phase;
the at least one source of each sensing location is configured to emit infrared radiation in a first hydrocarbon wavelength band, and in a second hydrocarbon wavelength band;
at least one of the first hydrocarbon wavelength band and the second hydrocarbon wavelength band resides within a region of the infrared spectrum in which the hydrocarbon phase exhibits absorption; and
the controller is configured to determine the output indicative of the concentration of water in dependence on determining a relative hydrocarbon response parameter in dependence on a third output signal from the at least one photodetector, indicative of infrared radiation received in the first hydrocarbon wavelength band, and a fourth output signal from the at least one photodetector, indicative of infrared radiation received in the second hydrocarbon wavelength band, and in dependence on determining a combined relative parameter in dependence on the relative water response parameter and the relative hydrocarbon response parameter.

19. An apparatus for determining an output indicative of a concentration of a substance in a multiphase flowable medium flowing in a flow direction through a conduit, the multiphase flowable medium comprising a plurality of phases, and the apparatus comprising:
a probe body configured to extend from a wall of the conduit, into the multiphase flowable medium flowing therethrough, the probe body defining a sensing location provided with:
at least one source configured to emit radiation from an infrared spectrum into the multiphase flowable medium in a first wavelength band and in a second wavelength band, each of the first wavelength band and the second wavelength band residing along a region of the infrared spectrum in which the substance exhibits absorption;
at least one photodetector configured to detect infrared radiation received from the at least one source via the multiphase flowable medium in the first wavelength band and the second wavelength band; and
a controller configured to determine an output indicative of a concentration of a substance in the multiphase flowable medium, based on determining a relative absorption parameter in dependence on a first output signal from the at least one photodetector, indicative of infrared radiation received in the first wavelength band, and a second output signal from the at least one photodetector, indicative of infrared radiation in the second wavelength band,
wherein the first wavelength band and the second wavelength band are each at different respective regions of the infrared spectrum on the same absorption peak of the substance.

20. (canceled)

21. A method of determining one or more parameters of a multiphase flowable medium flowing in a flow direction through a conduit, the multiphase flowable medium comprising at least a water phase, and the method comprising:
providing an apparatus comprising:
a probe body extending from a wall of the conduit and into the multiphase flowable medium flowing therethrough, the probe body defining a plurality of sensing locations, each for a different portion of the multiphase flowable medium and mutually spaced from any other one of the plurality of sensing locations in a direction having at least a component transverse to the flow direction;
at least one source configured to emit radiation from an infrared spectrum into the multiphase flowable medium in a first wavelength band and in a second wavelength band, each of the first wavelength band and the second wavelength band residing along a region of the infrared spectrum in which the substance exhibits absorption; and
at least one photodetector configured to detect infrared radiation received from the at least one source via the multiphase flowable medium in the first wavelength band and the second wavelength band;
controlling the at least one source of each sensing location to emit infrared radiation into the multiphase flowable medium at the respective sensing location;
receiving an output signal from the at least one photodetector of each sensing location; and
determining one or more parameters of the multiphase flowable medium in dependence on the received output signals.

22. A method of determining an output indicative of a concentration of a substance in a multiphase flowable medium flowing in a flow direction through a conduit, the multiphase flowable medium comprising a plurality of phases, and the method comprising:
providing an apparatus comprising:
a probe body extending from a wall of the conduit and into the multiphase flowable medium flowing therethrough, the probe body defining a plurality of sensing locations, each for a different portion of the multiphase flowable medium and mutually spaced from any other one of the plurality of sensing locations in a direction having at least a component transverse to the flow direction;
at least one source configured to emit radiation from an infrared spectrum into the multiphase flowable medium in a first wavelength band and in a second wavelength band, each of the first wavelength band and the second wavelength band residing along a region of the infrared spectrum in which the substance exhibits absorption;
at least one photodetector configured to detect infrared radiation received from the at least one source via the multiphase flowable medium in the first wavelength band and the second wavelength band; and
a controller configured to determine an output indicative of a concentration of a substance in the multiphase flowable medium, based on determining a relative absorption parameter in dependence on a first output signal from the at least one photodetector, indicative of infrared radiation received in the first wavelength band, and a second output signal from the at least one photodetector, indicative of infrared radiation in the second wavelength band;
wherein the first wavelength band and the second wavelength band are each at different respective regions of the infrared spectrum on the same absorption peak of the substance;
using the controller, controlling the at least one source to emit infrared radiation into the multiphase flowable medium in the first wavelength band and the second wavelength band;
receiving, from the at least one photodetector, a first output signal indicative of infrared radiation received in the first wavelength band, and a second output signal indicative of infrared radiation received in the second wavelength band; and
determining an output indicative of a concentration of a substance in the multiphase flowable medium, based on determining a relative absorption parameter in dependence on the first output signal and the second output signal.

23. A method of determining one or more parameters of a multiphase flowable medium flowing in a flow direction through a conduit, wherein:
the multiphase flowable medium comprises at least a water phase and a hydrocarbon phase, and
the method comprises:
receiving a plurality of concentration inputs, each indicative of a concentration of at least one substance of the multiphase flowable medium in the conduit at a respective position within the conduit, each position mutually spaced in a direction transverse to the flow direction;
outputting one or more parameters of the multiphase flowable medium in dependence on the plurality of concentration inputs.

24. The method of claim 23, wherein the one or more parameters include a phase amount indicator, indicative of an amount of at least one substance of the multiphase flowable medium flowing through the conduit.

25. The method of claim 23, further comprising:
using a controller, determining a gradient indicator indicative of an emulsion concentration gradient in response to the output indicative of a concentration of a substance in the multiphase flowable medium, and
determining an emulsion-oil phase change position and an emulsion-water phase change position in the conduit in response to the gradient indicator.
